SEQUENCE LISTING (7 sequences)

SEQ ID NO: 1 - DNA, 1500 bp, Aspergillus Fumigatus
  sig_peptide (164)..(214)
  CDS (215)..(1015)
gcagagtacgcgggggtctctcagatcccagagaaagaacactcagacccagtctttcgcttgctcgatcgatcccattcgtttggctcactcgaatctgtccatctatacagccacagttttagatacaactaatctctaatactacccttcgttggtcaaaatgcgtttctctgccctcctcgtcactctcggcctcaccggtgccctggccacgcccaccctggtctctcgtgaggcccctgccgtcggtgtcatctccgacatctcggcccagacctctgctctggcctccgccgtctcctcctacaacggtggtgacccctccgccgtcaagtccgcctctgagaagcttgtcagcaccatcaactccggtgtcgacaccgtcaagagcggccctgccctcagcaccgctgatgctctggctctgacctctcccgtccaggacctgaccaagcaggtcgagggcgtcatcgacgacctcatctccaagaaggacaagttcgtcgccgccaacgccggtggcactgtctacgaggacctcaaggcccagtacaccgctgccgacagcctggccaaggccatctccgccaaggtccctgagtccctctccgacatcgccgctcagctgtccgctggcattaccgccgccattcagaagggtatcgacgcctacaaggacgccgccagctctaccggcactgcttcctcttctgcccctgccactgagaccgcgaccgccaccgagacctccactgccactggcaccgtcaccgagacggccacttccacccctgtcatccccaccggtaccgcgtctggcagcgcctccgctaccccctccaccaccgctacccccaccacgggcggctccggctccggctccggctccagcactggtactgccactgcctccaccagcaccaacctcctctccactggcgccgccagcaaggagcacttcagctactccctcggcggtgccgtcgtcgcggccgccatcgccgtcgctctctaagcgccctctttctgactttctccggctccggctccggctaccagcactggtacttgccacttgcctccaccagcaccaacctcctctccactggcgccgccagcaaggagcacttcagctactccctcggcggtgccgtcgtcgcggccgccatcgccgtcgctctctaagcgccctctttctgactttgcctattggctctctgagtcaaacacaagtcggacggcggacggcggagagcaacgcaatgatgcgatgggaacgttgatgacgatcattcatgaggcaagaccagggttccaacgatcggttcatgaaagcgttatgcgagaacaatggccgatgacaccatagggcttatcggctttccatccgatctttgtacttgttagtccaccgtcgggtccgggtcgccggcttggatccgtctgtacacatatatagttttaataattgatatctctgtataaaaaaaaaaaaaa

SEQ ID NO: 2 - protein, 267 aa, Aspergillus Fumigatus
TPTLVSREAPAVGVISDISAQTSALASAVSSYNGGDPSAVKSASEKLVSTINSGVDTVKSGPALSTADALALTSPVQDLTKQVEGVIDDLISKKDKFVAANAGGTVYEDLKAQYTAADSLAKAISAKVPESLSDIAAQLSAGITAAIQKGIDAYKDAASSTGTASSSAPATETATATETSTATGTVTETATSTPVIPTGTASGSASATPSTTATPTTGGSGSGSGSSTGTATASTSTNLLSTGAASKEHFSYSLGGAVVAAAIAVAL

SEQ ID NO: 3 - protein, 17 aa, Aspergillus fumigatus
  SIGNAL (1)..(17)
MRFSALLVTLGLTGALA

SEQ ID NO: 4 - DNA, 25 bp, Aspergillus fumigatus
  prim_transcript (1)..(25)
tgtcggagagggactcagggacctt

SEQ ID NO: 5 - DNA, 25 bp, Aspergillus fumigatus
  prim_transcript (1)..(25)
ggcgacgaacttgtccttcttggag

SEQ ID NO: 6 - DNA, 25 bp, Aspergillus fumigatus
  prim_transcript (1)..(25)
ccgtctcctcctacaacggtggtga

SEQ ID NO: 7 - DNA, 24 bp, Aspergillus fumigatus
  prim_transcript (1)..(24)
ctcatctccaagaaggacaagttc

(19) United States
(12) Patent Application Publication
(10) Pub. No.: US 2002/0034751 A1
(43) Pub. Date: Mar. 21, 2002

(54) Aspergillus fumigatus antigenic protein 1

(76) Inventors: Kwok-Yung Yuen, Hong Kong (HK); Liang Cao, Hong Kong (HK)

Correspondence Address: Stephen D. Scanlon, Esq., Jones, Day, Reavis & Pogue, North Point, 901 Lakeside Avenue, Cleveland, OH, 44114, US

(21) Appl. No.: 09/852,555

(22) Filed: May 10, 2001

Related U.S. Application Data
(60) Provisional application No. 60/203,322, filed on May 10, 2000.

(51) Int. Cl. C12Q 1/68; C07H 21/04; C12N 1/15; C12P 21/02

(52) U.S. Cl. 435/6; 435/69.3; 435/320.1; 536/23.7; 435/254.11

(57) ABSTRACT

Disclosed is an Aspergillus fumigatus AFAP1 polypeptide and DNA (RNA) encoding such AFAP1 polypeptide. Also provided is a procedure for producing such polypeptide by recombinant DNA techniques and a procedure for generating antibodies against the polypeptide. Also disclosed is a method of using such polypeptide and the antibodies against it for the diagnosis of deep infections of Aspergillus fumigatus by detecting the presence of the specific antibodies as well as the AFAP1 protein antigen in clinical specimens taken from suspected patients. Also provided are methods of using the AFAP1 DNA(RNA) or protein sequence to identify and to clone its homologous genes from other Aspergillus species. Therefore, the identification of AFAP1 homologous genes from other pathogenic fungi are made possible with this invention. Also described is a therapeutic regimen using the antibodies against Aspergillus infection. Also provided is a method of immunization against the infection of Aspergillosis.

### RELATED APPLICATION

This application claims priority from U.S. Provisional Application Ser. No. 60/203,322 entitled, “Aspergillus Fumigatus Antigenic Protein 1”, filed May 10, 2000.

### TECHNICAL FIELD OF INVENTION

This invention relates to a newly characterised complete polynucleotide (gene), a polypeptide encoded by such polynucleotide, the use of such polynucleotide and polypeptide, as well as the production of such polypeptide.

### BACKGROUND OF THE INVENTION

Invasive aspergillosis is the most important cause of mortality in patients with haemic malignancies. The incidence is about 15% in patients suffering from acute leukaemia, 7.4% in bone marrow transplant, and 2.5% in solid organ transplant or chemotherapy for haemic malignancies or autologous marrow transplants. Up to 12% of AIDS patients and 40% of patients with chronic granulomatous disease could be affected by this infection. The mortality rate of invasive aspergillosis is 82% even when treated. In contrast, immunocompetent patients are rarely affected by invasive aspergillosis but 10-15% of patients with cavitating lung diseases, especially old tuberculosis, suffer from aspergilloma. This form of aspergillosis is rarely symptomatic except when the spherical mass of hyphae occupying the cavity erodes into blood vessels in the wall and leads to haemoptysis. Of all the known Aspergillus species, Aspergillus fumigatus is the most common species associated with human disease.

The diagnosis of aspergilloma depends on suggestive chest radiographic changes including spherical mass surrounded by a radiolucent crescent, with or without adjacent pleural thickening, and a high titre of antibodies as demonstrated by immunoprecipitation with crude and poorly standardized antigenic extract from Aspergillus fumigatus. Unlike a closely related fungus, Penicillium marneffei, no antibody test kit using recombinant antibody is currently available for making a diagnosis.

No pathognomonic clinical features could be found to identify cases of invasive aspergillosis early enough to guide specific treatment with a very toxic antifungal agent called amphotericin B. The gold standard for making a diagnosis is to get a positive culture of Aspergillus fumigatus and to demonstrate histological evidence of mycelial invasion in tissue biopsy. Due to the very sick nature of these patients, and often the presence of bleeding diathesis, tissue biopsy is often not possible or acceptable by patients. Currently, early specific antifungal therapy is justified if (i) a compatible clinical setting; (ii) a suggestive radiological feature; (iii) culture and microscopic evidence of Aspergillus species in clinical specimens such as sputum or bronchoalveolar lavage; or (iv) detection of Aspergillus antigen or DNA in serum are present.

Detection of Aspergillus DNA by polymerase chain reaction is difficult to control for false positivity because fungal spores are so ubiquitous and fungal DNA has been reported to contaminate the enzymes used for fungal DNA extraction. Presence of Aspergillus DNA in respiratory secretion could mean low level colonisation rather than invasive disease by Aspergillus species. Moreover, Aspergillus is rarely cultured from blood of patients even at the terminal stage of invasive aspergillosis.

Detection of antibody is less useful than detection of antigen in the diagnosis of invasive aspergillosis because immunosuppressed patients may fail to mount a good humoral response and low titres in normal hosts may confound the specificity of an antibody assay. At present, commercial kits for antigen detection assay using monoclonal antibody against the galactomannan antigen extract is available for clinical use. Using the latex agglutination format (Pastorex®), the sensitivity is 25-70% and the specificity is 90-100%. Whereas if sandwich ELISA Platella® is used, the sensitivity is 80-100% and specificity is 80-90%. No antigen detection kit based on recombinant antigens of Aspergillus is presently available. Of the 13 reported recombinant proteins of Aspergillus fumigatus, all except one with known functions are enzymes including RNase, superoxide dismutase, serine protease, aspartic protease, metalloprotease, dipeptidyl peptidase and catalase. The exception is a peroxisomal protein. None of these has been reported clinically to be useful for the diagnosis of aspergillosis or invasive aspergillosis.

### SUMMARY OF THE INVENTION

We previously discovered a novel mannoprotein PMAP-1 in Penicillium marneffei and successfully used it for the serodiagnosis and clinical management of patients with this fungal infection. Aspergillus is phylogentically related to Pencillium. This patent is based on a novel Aspergillus mannoprotein which is closely related to the PMAP-1. This novel gene and protein are similarly used in the diagnosis of aspergillosis.

Recombinant antibody and antigen detection tests are the ideal tests for detecting the presence of Aspergillus due to their generally higher sensitivity, specificity and reproducibility. Moreover, recombinant antigens and generated antibodies are easy to standardize. The invention of such a gene and polypeptide for the manufacture of such detection tests would allow for an early and rapid diagnosis leading to early and specific antifungal therapy. This would improve the mortality of the disease and minimize the expense and side effects of empirical antifungal therapy.

The present invention solves the problem of diagnosing deep or systemic infection of fungus Aspergillus fumigatus by identifying one of the immunogenic proteins produced by this fungus and by cloning the gene that encodes this protein. This finding derives from our previous patented gene PMAP-1 and its protein sequence which is found to share homology with an unknown incomplete DNA fragment of Aspergillus fumigatus in the public database GenBank. This gene was cloned and completely sequenced. The recombinant protein of this gene as indicated in this invention has been shown to be of significant value in the diagnosis of deep or systemic Aspergillus fumigatus infection. The results in this invention indicate:

1. AFAP1 is a novel gene that shares 34% of the amino acid sequence with PMAP-1 at the conserved stretch of 141 amino acid and 80% of its complete protein sequence with an incomplete DNA sequence in the public gene database.

2. The gene encodes a highly immunogenic protein for this fungus. The acute sera of immunocompetent and some immunosuppressed patients with documented biopsy or culture positive Aspergillus fumigatus infection showed extremely high levels of specific antibodies against this protein comparable to that of immunized animals.

3. Patients with aspergilloma or deep Aspergillus infection showed elevated levels of antibody when compared with the normal blood donors.

4. No cross reactivity with sera from other systemic mycoses patients was observed under high stringency wash condition, indicating the high specificity of the test.

5. AFAP1 protein was expressed as a GST-AFAP1 fusion protein. The fusion protein was purified from E. coli bacterial cells and specific antibodies were generated by immunizing animals.

6. An ELISA based anti-AFAP1 antibody test using purified AFAP1 protein was produced and used for the detection of specific antibodies against AFAP1 protein in the Aspergillus fumigatus infected patients' sera.

7. The Anti-AFAP1 antibody test is effective for the detection of anti-AFAP1 antibodies in patients with invasive aspergillosis and aspergilloma. The test is sensitive and specific.

8. An ELISA-based AFAP1 antigen test using two types of anti-AFAP1 antibodies for a sandwich assay for AFAP1 protein.

9. The AFAP1 antigen test can be detected in the culture media of Aspergillus fumigatus cells.

10. AFAP1 protein can be detected in serum samples of patients with invasive aspergillosis.

In accordance with one aspect of the present invention, there is provided a novel, mature polypeptide, as well as diagnostically or therapeutically useful fragments, analogues and derivatives thereof. The polypeptide of the present invention is of Aspergillus fumigatus origin.

In accordance with another aspect of the present invention, there are provided isolated nucleic acid molecules encoding the polypepides of the present invention, including mRNAs, DNAs, cDNAs, genomic DNA, as well as diagnostically or therapeutically useful fragments, analogues and derivatives thereof.

In accordance with still another aspect of the present invention, there are provided processes for producing such polypeptide by recombinant techniques comprising culturing recombinant prokaryotic and/or eukaryotic host cells, containing a nucleic acid sequence encoding a polypeptide of the present invention, under conditions promoting expression of the protein and subsequent recovery of the protein.

In accordance with yet a further aspect of the present invention, there is provided a process for utilizing such polypeptide, or polynucleotide encoding such polypeptide for diagnostic purposes. For example, the diagnosis of systemic infection by Aspergillus fumigatus. An immunoassay can be established using the recombinant protein for serological testing of the presence of specific antibodies in the suspected patient's blood and urine which would indicate the specific infection.

In accordance with yet another aspect of the present invention, there are provided methods of producing antibodies against such polypeptides. The antibodies can be polyclonal or monoclonal.

In accordance with yet another aspect of the present invention, the specific antibodies can be used for the detection of specific antigen of AFAP1 nature in specimens from suspected patients, including blood, urine, cerebrospinal fluid, and tissue biopsies. The presence of the protein antigen is indicative of current infection by Aspergillus fumigatus.

In accordance with yet another aspect of the present invention, the diagnostic test for antigen may be used for the evaluation of the patient's response to anti-fungal treatments. A decrease in the antigen level in blood or urine can be an indicator of adequate response of patients during treatment.

In accordance with yet another aspect of the present invention, the diagnostic test for antigenaemia may be used for the detection of potential relapse of the original pathogenic fungus after discontinuing anti-fungal treatment.

In accordance with yet another aspect of the present invention, the antibodies against AFAP1 protein may be used for the passive immunization or therapeutic purposes against the infection.

In accordance with yet another aspect of the present invention, there is provided a reagent for immunization which may be used to prevent the infection of Aspergillus fumigatus for the people at high risk.

In accordance with another aspect of the present invention, there are provided nucleic acid probes comprising nucleic acid molecules of sufficient length to specifically hybridize to nucleic acid sequences of the present invention. The probes can be used for the identification of homologous genes from other Aspergillus species from either genomic DNA or cDNA libraries.

These and other aspects of the present invention should be apparent to those skilled in the art from the teachings herein.

### BRIEF DESCRIPTION OF THE DRAWINGS

The file of this patent contains at least one drawing executed in color. Copies of this patent with color drawing(s) will be provided by the Patent and Trademark Office upon request and payment of the necessary fee.

The following drawings are illustrative of embodiments of the invention and are not meant to limit the scope of the invention as encompassed by the claims.

FIG. 1 is the cDNA sequence for the AFAP1 protein and the corresponding deduced amino acid sequence of the polypeptide of the present invention (SEQ ID NO:1). The standard one letter abbreviations for amino acids are used. The N-terminal cleavable signal peptide of 17 amino acids is underlined. A serine/threonine rich region is shown in italics;

FIG. 2 is an illustration of the AFAP1 protein sequence motifs indicative of a cell wall protein of AFAP1 and its comparison with other cell wall proteins in yeast Saccharomyces cerevisiae, Candida albicans and Penicillium marneffei. The protein contains several structure motifs that are common for fungal cell wall proteins, including a signal peptide at the N-terminus and a GPI membrane anchor motif at the C-terminus;

FIG. 3 is a photograph of a gel after in-vitro transcription, translation, and SDS-PAGE gel electrophoresis separation of the polypeptide of the present invention. Lane 1: in-vitro translated AFAP1 crude extract; Lanes 2, 3: immunoprecipitation of in-vitro translated AFAP1 by guinea pig serum immunized against whole cell Aspergillus fumigatus and AFAP1 fusion protein respectively;

FIG. 4 is a photograph of a gel showing that the protein produced in-vitro can be recognized specifically by sera from patients that have invasive infection of Aspergillus fumigatus, but not from sera taken from patients with a different type of systemic infection by Candida albicans and Penicillium marneffei. Lane 1. in-vitro translated AFAP1; Lanes 2-20: immunoprecipitation of in-vitro translated AFAP1 by sera (Lanes 2, 12: immune guinea pig sera against AFAP1; Lanes 3-6: patients with aspergillosis; Lanes 7-10, 17-20: normal blood donors; Lanes 13-14: patients with candidaemia; Lanes 16-17: patients with penicilliosis marneffei);

FIG. 5a is a map of an expression plasmid construct that contains AFAP1 fused in frame with glutathione S-transferase (GST) allowing the expression and purification of GST-AFAP1 fusion protein;

FIG. 5b is a map of a eukaryotic expression plasmid construct that contains AFAP1 with a signal peptide;

FIG. 6 is a photograph of a Western Blot showing the recombinant AFAP1-GST fusion protein produced and purified from E. coli bacterial cell and similarly in eukaryotic cells. Lane 1: The sequence coding amino acid residues 19 to 284 of the AFAP1 protein was expressed in the prokaryotic vector pGEX2T; Lane 2: Supernatant from 293 cells which were transfected with the eukaryotic vector pSecTag2 encoding MP1 (recombinant protein of Penicillium marneffei); Lane 3: Supernatant from 293 cells which were transfected with eukaryotic vector pcDNA3 encoding full length sequence of AFAP1; Lane 4: 293 cell lysate were transfected with eukaryotic vector pcDNA3 encoding full length sequence of AFAP;

FIG. 7a is a photograph of a microscope slide from an indirect immunofluorescence experiment. Indirect immunofluorescent staining of Aspergillus mold cells for AFAP1 by monospecific guinea pig anti-sera against AFAP1 shows that the protein is specifically located in the mycelial wall. AFAP1 is seen as emitting a green fluorescence whereas the mycelial wall is seen as emitting a red fluorescence;

FIG. 7b is a photograph of a microscope slide from an indirect immunofluorescence experiment of a positive control using guinea pig anti-sera against whole cells of Aspergillus fumigatus. The AFAP 1 is seen as emitting a diffuse green fluorescence;

FIG. 7c is a photograph of a microscope slide from an indirect immunofluorescence experiment of a negative control using non-immune guinea pig sera against whole cells of Aspergillus fumigatus. The absence of green fluorescence shows a negative reaction;

FIG. 7d is an electron microscope photograph showing that immunogold staining of Aspergillus fumigatus cells with specific rabbit anti-AFAP1 antibody reveals that the protein is specifically located at the inner layer of the mycelial wall throughout the entire cell including the tip and septum;

FIG. 7e is an electron microscope photograph showing that immunogold staining of Aspergillus fumigatus cells with specific rabbit anti-AFAP1 antibody reveals that the protein is specifically located at the inner layer of the mycelial wall throughout the entire cell including the tip and septum;

FIG. 7f is an electron microscope photograph of a negative control experiment showing that negative control immunogold staining of Aspergillus fumigatus mycelia reveals no specific staining of the cell;

FIG. 8 is a graph depicting the detection of specific antibodies in patients with invasive aspergillosis and aspergilloma with an ELISA-based serological test using purified recombinant AFAP1 protein;

FIG. 9a is a graph depicting the specific detection of AFAP1 protein in Aspergillus fumigatus culture media with an ELISA-based antigen test for AFAP1 antigen using specific antibodies against AFAP1 recombinant protein;

FIG. 9b is a graph showing that the AFAP1 protein is found in the supernatant of the cell culture media in which the Aspergillus fumigatus mold cells are incubated but the AFAP1 protein is not found in the supernatant of the cell culture media of other fungal cultures that were tested; and

FIG. 10 is a graph showing that AFAP1 protein antigen can be specifically detected by ELISA in the serum samples of immunocompromised patients with invasive aspergillosis but not in other patient groups.

### DETAILED DESCRIPTION OF THE INVENTION

The term “gene” means the segment of DNA involved in producing a polypeptide chain. It includes regions preceding and following the coding region (leader and trailer), as well as intervening sequences (introns) between individual coding segments (exons).

AFAP1 Gene

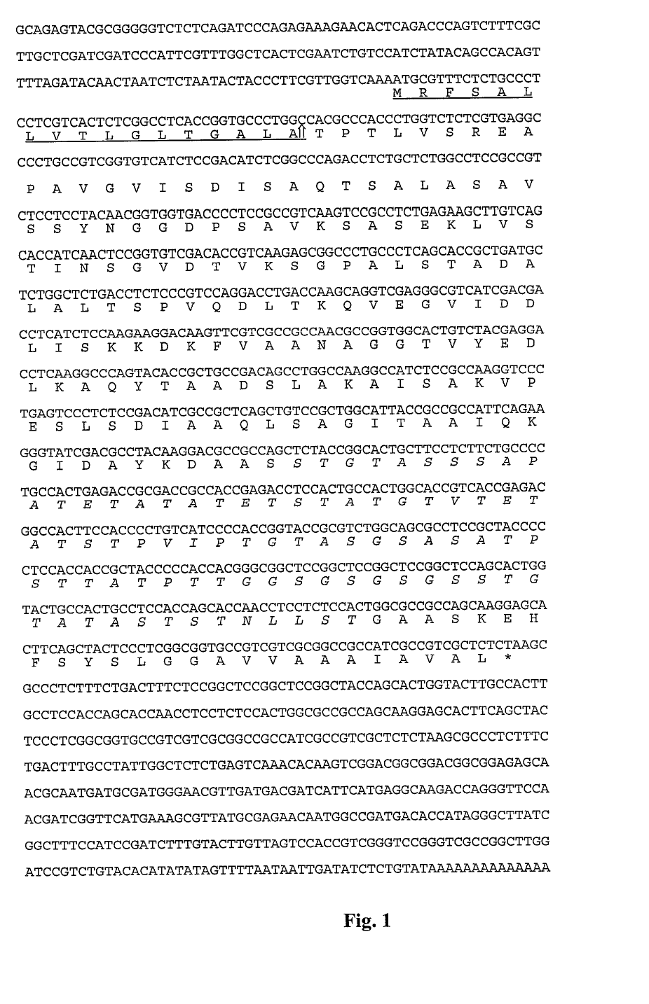

In accordance with one aspect of the present invention, there are provided isolated nucleic acid molecules (polynucleotides) which encode for the mature polypeptides having the deduced amino acid sequence of FIG. 1 for polypeptides which have fewer amino acid residues than those showing in FIG. 1.

A polynucleotide encoding a polypeptide of the present invention may be obtained from cDNA or genomic DNA libraries from Aspergillus fumigatus. The polynucleotide of this invention was discovered in a cDNA library derived from Aspergillus fumigatus by RACE PCR and an antibody-based, highly selective process as described in Example 1.

This polynucleotide encoding a polypeptide of the present invention can be obtained from cDNA or genomic DNA libraries from Aspergillus fumigatus using polynucleotide probes derived from SEQ ID NO:1 of this invention.

Alternatively, it can be obtained by the amplification of either cDNA or genomic DNA from Aspergillus fumigatus through polymerase chain reaction (PCR) using appropriate polynucleotide primers derived from SEQ ID NO:1.

It is a complete gene with a single open reading frame and stop codon. BLAST analysis (NCBL National Library of Medicine) of both AFAP1 nucleotide and protein sequences against GCG data base failed to identify any significant homology with any existing gene but has 34% amino acid at the conserved region of 141 amino acid within the protein of Penicillium marneffei called PMAP-1. AFAP1 contains an open reading frame encoding a protein of 284 amino acid residues.

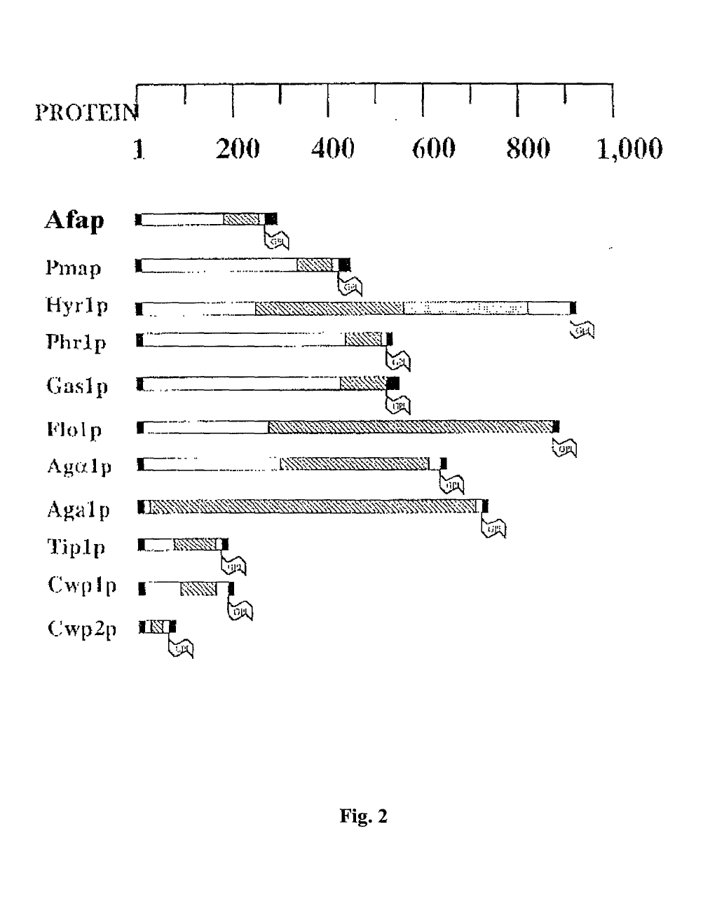

It is apparent that the protein contains several structure motifs that are common for fungal cell wall proteins, including a signal peptide at the N-terminus (SEQ ID NO: 3) and a GPI membrane anchor motif at the C-terminus, suggesting that AFAP1 is localized on yeast cell wall (FIG. 2). In addition, AFAP1 contains a serine and threonine rich region at the C-terminal half, suggesting that the protein is glycosylated.

Once processed, the matured AFAP1 polypeptide of the present invention is 267 amino acid residues (SEQ ID NO: 2), as shown in FIG. 2.

The polynucleotide of the present invention may be in the form of RNA or in the form of DNA, which DNA includes cDNA, genomic DNA, and synthetic DNA. The DNA may be double-stranded or single-stranded, and if single-stranded may be the coding strand or non-coding (anti-sense) strand. The coding sequence which encodes for the mature polypeptide may be identical to the sequence shown in FIG. 1 or that of the deposited clone may be a different coding sequence which coding sequence, as a result of the redundancy or degeneracy of the genetic code, endorsed the same mature polypeptide as the DNA of FIG. 1, or the deposit DNA.

The term “polynucleotide encoding a polypeptide” encompasses a polynucleotide which includes only coding sequence for the polypeptide as well as a polynucleotide which includes additional coding and/or non-coding sequence.

The present invention further relates to variants of the hereinabove described polynucleotides which encode for fragment, analogues, and derivatives of the polypeptide having the deduced amino acid sequence of FIG. 1. The variant of the polynucleotide may be a naturally occurring allelic variant of polynucleotide or a non-naturally occurring variant of the polynucleotide.

Thus, the present invention includes polynucleotides encoding the same mature polypeptide as shown in FIG. 1 or the same mature polypeptide encoded by the cDNA of the deposited clone, as well as variants of such polynucleotides in which variants encode for a fragment, derivative or analogue of the polypeptide of FIG. 1, or the polypeptide encoded by the cDNA of the deposited clone. Such nucleotide variants include deletion variants, substitution variants and addition or insertion variants.

As hereinabove indicated, the polynucleotide may have a coding sequence which is a naturally occurring allelic variant of the coding sequence shown in FIG. 1 or of the coding sequence of the deposited clone. As known in the art, an allelic variant is an alternate form of a polynucleotide sequence which may have a substitution, deletion, or addition of one or more nucleotides, which does not substantially alter the function of the encoded polypeptide.

The polynucleotides of the present invention may also have the coding sequence fused in frame to a marker sequence which allows for purification of the polypeptide of the present invention. The marker sequence may be a hexa-histidine tag supplied by a pQE30 vector to provide for purification of the mature polypeptide fused to the marker in the case of a bacterial host, or for example, the marker sequence may be a haemagglutinin (HA) tag when a mammalian host, e.g. COS-7 cells, is used. The HA tag corresponds to an epitope derived from the influenza haemagglutinin protein (Wilson, T., et al. Cell 1984; 3:757).

Fragments of the full length gene of the present invention may be used as a hybridization probe for a cDNA library to isolate the full length cDNA and to isolate other cDNAs which have a high sequence similarity to the gene. Probes of this type preferably have at least 30 bases and may contain, for example, 50 or more bases. The probe may also be used to identify a cDNA clone or corresponding to a full length transcript and a genomic clone or clones that contain the complete gene including regulatory promoter regions, exons, and introns. An example of a screen comprises isolating the coding region of the known gene by using the known DNA sequence to synthesize an oligonucleotide probe. Labelled oligonucleotides having a sequence complementary to that of the gene of the present invention are used to screen libraries of pathogenic fungi's cDNA, genomic DNA or mRNA to determine which members of the library the probe hybridizes to.

The present invention further relates to polynucleotides which hybridize to the hereinabove-described sequences if there is at least 70%, preferably at least 90%, and more preferably at least 95% identity between the sequences. The present invention particularly relates to polynucleotides which hybridize under stringent conditions to the hereinabove-described polynucleotides. As herein used, the term “stringent conditions” means hybridization will occur only if there is at least 95% and preferably at 97% identity between the sequences. The polynucleotides which hybridize to the hereinabove described polynucleotides in a preferred embodiment encode polypeptides which either retain substantially the same biological function or activity as the mature polypeptide encoded by the cDNAs of FIG. 1.

Alternatively, the polynucleotide may have at least 20 bases, preferably 30 bases, and more preferably at least 50 bases, which hybridize to a polynucleotide of the present invention and which has an identity thereto, as hereinabove described, and which may or may not retain activity. For example, such polynucleotides may be employed as probes for the polynucleotide of FIG. 1, for recovery of the polynucleotide, as a diagnostic probe, or as a PCR primer.

AFAP Polypeptides

The present invention further relates to a polypeptide which has the deduced amino acid sequence of FIG. 1 or which has the amino acid sequence encoded by the deposited cDNA, as well as fragments, analogues and derivatives of such a polypeptide.

The terms “fragment”, “derivative” and “analogue” when referring to the polypeptide of FIG. 1, or that encoded by the deposited cDNA, mean any part of the polypeptide proteins as shown in FIG. 1.

The polypeptides of the present invention may be recombinant polypeptides, natural polypeptides or synthetic polypeptides, preferably recombinant polypeptides.

The fragment, derivative, or analogue of the polypeptide of FIG. 1, or that encoded by the deposited cDNA may be (i) one in which one or more of the amino acid residues are substituted with a conserved or non-conserved amino acid residue (preferably a conserved amino acid residue) and such substituted amino acid residue may or may not be one encoded by the genetic code; or (ii) one in which one or more of the amino acid residues includes a substitute group; or (iii) one in which the mature polypeptide is fused with another compound, such as a compound to increase the half-life of the polypeptide (for example, polyethylene glycol); or (iv) one in which the additional amino acids are fused to the mature polypeptide; or (v) one which comprises fewer amino acid residues than shown in FIG. 1. Such fragments, derivatives and analogues are deemed to be within the scope of those skilled in the art from the teachings herein.

The polypeptides and polynucleotides of the present invention are preferably provided in an isolated form, and preferably are purified to homogeneity.

The term “isolated” means that the material is removed from its original environment (e.g. the natural environment if it is naturally occurring). For example, a naturally-occurring polynucleotide or polypeptide present in a living organism is not isolated, but the same polynucleotide or polypeptide, separated from some or all of the coexisting materials in the natural system is isolated. Such polynucleotides could be part of a vector and/or such polynucleotides or polypeptides could be part of a composition and still be isolated in that such vector or composition is not part of its natural environment.

The polypeptides of the present invention include the polypeptide of FIG. 1 (in particular the mature polypeptide) as well as polypeptides which have at least 70% similarity (preferably at least 70% identity) to the polypeptide of FIG. 1, and more preferably at least 90% similarity (more preferably at least 95% identity) to the polypeptide of FIG. 1, and still more preferably at least 95% similarity (still more preferably at least 95% identity) to the polypeptide of FIG. 1, and also include portions of such polypeptides with such portion of the polypeptide generally containing at least 30 amino acids, and more preferably at least 50 amino acids.

As known in the art, “similarity” between two polypeptides is determined by comparing the amino acid sequence and its conserved amino acid substitutes of one polypeptide to the sequence of a second polypeptide.

Fragments or portions of the polypeptides of the present invention may be employed for producing the corresponding full-length polypeptide by peptide synthesis. Therefore, the fragments may be employed as intermediates for producing the full-length polypeptides. Fragments or portions of the polynucleotides of the present invention may be used to synthesize full-length polynucleotides of the present invention.

The present invention also relates to vectors which include polynucleotides of the present invention, host cells which are genetically engineered with vectors of the invention and the production of polypeptides of the invention by recombinant techniques.

Host cells are genetically engineered (transduced, transformed or transfected) with the vectors of this invention which may be, for example, a cloning vector or an expression vector. The vector may be, for example, in the form of a plasmid, a viral particle, a phage, etc. The engineered host cells can be cultured in conventional nutrient media modified as appropriate for activating promoters, selecting transformants or amplifying the AFAP1 genes of the present invention. The culture conditions, such as temperature, pH and the like, are those previously used with the host cell selected for expression, and will be apparent to the ordinarily skilled artisan.

The polynucleotides of the present invention may be employed for producing polypeptides by recombinant techniques. Thus, for example, the polynucleotide may be included in any one of a variety of expression vectors for expressing a polypeptide. Such vectors include chromosomal, non-chromosomal and synthetic DNA sequences, e.g., derivatives of SV40, bacterial plasmids, phage DNA, baculovirus, yeast plasmids, vectors derived from combinations of plasmids and phage DNA, viral DNA such as vaccinia, adenovirus, fowl pox virus, and pseudorabies. However, any other vector may be used as long as it is replicable and viable in the host.

The appropriate DNA sequence may be inserted into the vector by a variety of procedures. In general, the DNA sequence is inserted into an appropriate restriction endonuclease site(s) by procedures known in the art. Such procedures and others are deemed to be within the scope of those skilled in the art.

The DNA sequence in the expression vector is operatively linked to an appropriate expression control sequence(s) (promoter) to direct mRNA synthesis. As representative examples of such promoters, there may be mentioned: LTR or SV40 promoter, the E. coli. lac or trp, the phage lambda P_{L} promoter and other promoters known to control expression of genes in prokaryotic or eukaryotic cells or their viruses. The expression vector also contains a ribosome binding site for translation, initiation, and a transcription terminator. The vector may also include appropriate sequences for amplifying expression.

In addition, the expression vectors preferably contain one or more selectable marker genes to provide a phenotypic trait for selection of transformed host cells such as dihydrofolate reductase or neomycin resistance for eukaryotic cell culture, or such as tetracycline or ampicillin resistance in E. coli.

The vector containing the appropriate DNA sequence as hereinabove described, as well as an appropriate promoter or control sequence, may be employed to transform an appropriate host to permit the host to express the protein. As representative examples of appropriate hosts, there may be mentioned: bacterial cells such as E. coli, Streptomyces, Salmonella typhimurium, fungal cells such as yeast, insect cells such as Drosophila S2 and Spodoptera Sf9, animal cells such as CHO, COS or Bowes melanoma, adenoviruses, plant cells, etc. The selection of an appropriate host is deemed to be within the scope of those skilled in the art from the teachings herein.

More particularly, the present invention also includes recombinant constructs comprising one or more of the sequences as broadly described above. The constructs comprise a vector, such as a plasmid or viral vector, into which a sequence of the invention has been inserted in a forward or reverse orientation. In a preferred aspect of this embodiment, the construct further comprises regulatory sequences, including for example, a promoter, operably linked to the sequence. Large numbers of suitable vectors and promoters are known to those of skill in the art and are commercially available. The following vectors are provided by way of example. Bacterial: pQE70, pQE60, pQE-30 (Qiagen), pBS, pD10, phagescript, psiX174, pBluescript SK, pBSKS, pNH8A, pNH16a, pNH18A, pNH46A (Stratagene); ptrc99a, pKK223-3, pKK233-3, pDR540, pRIT5 (Pharmacia). Eukaryotic: pWLNEO, pSV2CAT, pOG44, pXT1, pSG (Stratagene) pSVK3, pBPV, pMSG. pSVL (Pharmacia). However, any other plasmid or vector may be used as long as they are replicable and viable in the host.

Promoter regions can be selected from any desired gene using CAT (chloramphenicol transferase) vectors or other vectors with selectable markers. Two appropriate vectors are pKK232-8 and pCM7. Particular named bacterial promoters include lac, lacZ, T3, T7, gpt, lambda P_{R}, P_{L}, and trp. Eukaryotic promoters include CMV immediate early, HSV thymidine kinase, early and late SV40, LTRs from retrovirus, and mouse metallothionein-L. Selection of the appropriate vector and promoter is well within the level of ordinary skill in the art.

In a further embodiment, the present invention relates to host cells containing the above-described constructs. The host cell can be a higher eukaryotic cell, such as a mammalian cell, or a lower eukaryotic cell, such as a yeast cell. The host cell can be a prokaryotic cell, such as a bacterial cell. Introduction of the construct into the host cell can be effected by calcium phosphate transfection, DEAE-dextran mediated transfection, or electroporation (Davis, L., Dibner, M., Battey, I. Basic Methods in Molecular Biology (1986)).

The constructs in host cells can be used in a conventional manner to produce the gene product encoded by the recombinant sequence. Alternatively, the polypeptides of the invention can be synthetically produced by conventional peptide synthesizers.

Mature proteins can be expressed in mammalian cells, yeast, bacteria, or other cells under the control of appropriate promoters. Cell-free translation systems can also be employed to produce such proteins using RNAs derived from the DNA constructs of the present invention. Appropriate cloning and expression vectors for use with prokaryotic and eukaryotic hosts are described by Sambrook et al., Molecular Cloning: A Laboratory Manual, Second Edition, Cold Spring Harbor, N.Y, (1989).

Transcription of the DNA encoding the polypeptides of the present invention by higher eukaryotes is increased by inserting an enhancer sequence into the vector. Enhancers are cis-acting elements of DNA, usually from about 10 to 300 bp, that act on a promoter to increase its transcription. Examples include the SV40 enhancer on the late side of the replication origin bp 100 to 270, a cytomegalovirus early promoter enhancer, the polyoma enhancer on the late side of the replication origin, and adenovirus enhancers.

Generally, recombinant expression vectors will include origins of replication and selectable markers permitting transformation of the host cell, e.g. the ampicillin resistance gene of E. coli and S. cerevisiae TRP1 gene, and a promoter derived from a highly-expressed gene to direct transcription of a downstream structural sequence. Such promoters can be derived from operons encoding glycolytic enzymes such as 3-phosphoglycerate kinase (PGK), acid phosphatase, or heat shock proteins, among others. The heterologous structural sequence is assembled in appropriate phase with translation, initiation and termination sequences, and preferably, a leader sequence capable of directing secretion of translated protein into the periplasmic space or extracellular medium. Optionally, the heterologous sequence can encode a fusion protein including an N-terminal identification peptide imparting desired characteristics, e.g. stabilization or simplified purification of expressed recombinant product.

Useful expression vectors for bacterial use are constructed by inserting a structural DNA sequence encoding a desired protein together with suitable translation initiation and termination signals in operable reading phase with a functional promoter. The vector will comprise one or more phenotypic selectable markers and an origin of replication to ensure maintenance of the vector, and if desirable, to provide amplification within the host. Suitable prokaryotic hosts for transformation include E. coli, Bacillus subtilis, Salmonella typhimurium and various species within the genera Pseudomonas, Streptomyces, and Staphylococcus, although others may also be employed as a matter of choice.

As a representative but non-limiting example, useful expression vectors for bacterial use can comprise a selectable marker and bacterial origin of replication derived from commercially available plasmids comprising genetic elements of the well known cloning vector pBR322 (ATCC 37017). Such commercial vectors include, for example, pKK223-3 (Pharmacia Fine Chemicals, Uppsala, Sweden) and GEMl (Promega Biotec, Madison, Wis., USA). These pBR322 “backbone” sections are combined with an appropriate promoter and the structural sequence to be expressed.

Following transformation of a suitable host strain and growth of the host strain to an appropriate cell density, the selected promoter is induced by appropriate means (e.g. temperature shift or chemical induction), and cells are cultured for an additional period.

Cells are typically harvested by centrifugation, disrupted by physical or chemical means, and the resulting crude extract retained for further purification.

Microbial cells employed in expression of proteins can be disrupted by any convenient method, including freeze-thaw cycling, sonication, mechanical disruption, or use of cell lysing agents. Such methods are well know to those skilled in the art.

The polypeptides can be recovered and purified from recombinant cell cultures by methods including ammonium sulfate or ethanol precipitation, acid extraction, anion or cation exchange chromatography, phosphocellulose chromatography hydrophobic interaction chromatography, affinity chromatography, hydroxylapatite chromatography and lectin chromatography. Protein refolding steps can be used as necessary in completing configuration of the mature protein. Finally, high performance liquid chromatography (HPLC) can be employed for final purification steps.

The polypeptides of the present invention may be a naturally purified product, or a product of chemical synthetic procedures, or produced by recombinant techniques from a prokaryotic or eukaryotic host (for example, by bacterial, yeast, higher plant, insect, and mammalian cells in culture). Depending upon the host employed in a recombinant production procedure, the polypeptides of the present invention may be glycosylated or may be non-glycosylated. Polypeptides of the invention may also include an initial methionine amino acid residue.

Serological Test for Specific Antibodies Against AFAP1

The AFAP1 protein is produced in vitro and used for the testing of the presence of specific antibodies in serological samples from animals, before and after, Aspergillus fumigatus inoculation. As shown in Example 3, the AFAP1 polypeptide of FIG. 1 is produced by in vitro translation to give rise a polypeptide of 32 kDa. This specific polypeptide is recognized specifically by serum from the animals that were inoculated with Aspergillus fumigatus as demonstrated by a positive band of a 32 kDa protein on an SDS-PAGE gel after immunoprecipitation assay. The serum from those before fungal inoculation, however, did not precipitate the 32 kDa AFAP1 protein.

More importantly, the AFAP1 protein produced by in vitro translation can be specifically recognized by sera obtained from patients that have documented invasive aspergillosis or aspergilloma as described in Example 4. These patients can be either immunocompetent or immunosuppressed by steroid and chemotherapy. The levels of specific antibodies against AFAP1 protein antibody in immunocompetent patients are at very high levels comparable to that of the immunized Guinea pig. Those from immunosuppressed patients with invasive aspergillosis are somewhat lower but yet significantly higher than that of normal control people free from the infection. Thus, AFAP1 has been demonstrated to have utility for the diagnosis of aspergillosis by detecting the presence of specific antibodies against the AFAP1 protein in patients' sera.

In addition to its high sensitivity, this test using AFAP1 protein is also very specific. It is shown in Example 5 that sera from neither normal control (not infected) people, nor from those with some common systemic fungal infections, i.e. Candida albicans, Cryptococcus neoformans, or Penicillium marneffei, have any detectable levels of the specific antibody against AFAP 1.

AFAP1 polypeptides can be produced and isolated as described in Examples 6 and 7 and used for serological tests. The serological test for the presence of the specific antibody against AFAP1 can be of many different forms described herein and others within the limit of skill in the art, including but not limited to immunofluorescence, enzyme-linked immune assay, radioimmunoassay, complement fixation test, latex agglutination test, precipitation. immunodiffusion test, neutralization test, skin test and other methods derived from them.

For example, an enzyme linked lectinosorbent assay (ELLSA) (Coligan et al., Current Protocols in Immunology, 1(2), Chapter 6, 1991), would initially use the purified recombinant protein coated on a solid phase polystyrene dish. In addition, a reporter antibody is prepared against the human antibody. A detectable reagent is attached to the reporter antibody, such as radioactivity, fluorescence, or in this example, a horseradish peroxidase enzyme. First, the polystyrene dish is coated with the recombinant protein. Next, a blood sample is removed from a suspected patient and incubated in the dish during which time the specific human antibodies against AFAP1 can bind to any AFAP1 proteins coated onto the polystyrene dish. All unbound human antibodies are washed away with buffer. A reporter antibody linked to horseradish peroxidase is placed in the dish resulting in the binding of the reporter antibody to any human antibody bound to AFAP1. Unattached reporter antibody is then washed out. Peroxidase substrates are then added to the dish and the amount of color developed in a given time period is a measurement of the titer of AFAP1 antibody present in a given volume of patient sample. Examples 9 and 10 illustrate such work and the result of the work in diagnosis of Aspergillus fumigatus infections.

AFAP1 nucleic acid sequences and AFAP1 polypeptides may also be employed for in vitro purposes related to scientific research and diagnosis of the disease. For example, Polymerase Chain Reaction (PCR) can be used for the identification and diagnosis of the infection.

Specific Antibodies Against AFAP1

The polypeptides, their fragments or other derivatives, or analogues thereof, or cells expressing them can be used as an immunogen to produce antibodies thereto as described in example 8. These antibodies can be, for example, polyclonal or monoclonal antibodies. The present invention also includes chimeric, single chain, and humanized antibodies, as well as Fab fragments, or the product of an Fab expression library. Various procedures known in the art may be used for the production of such antibodies and fragments.

Antibodies generated against the polypeptide corresponding to a sequence of the present invention can be obtained by direct injection of the polypeptide into an animal or by administering the polypeptide to an animal. The antibody so obtained will then bind the polypeptide itself. In this manner, even a sequence encoding only a fragment of the polypeptide can be used to generate antibodies binding the whole native polypeptide. Such antibodies can then be used to isolate the polypeptide from tissue expressing that polypeptide. For preparation of monoclonal antibodies, any technique which provides antibodies produced by continuous cell line cultures can be used. Examples include the hybridoma technique, (Kohler and Mustein, 1975, Nature 256:495-497); the trioma technique; the human B-cell hybridoma technique (Kozbor et al. 1983, Immunology Today 4:72); and the EBV-hybridoma technique to produce human monoclonal antibodies (Cole. et al. 1985, in Monoclonal Antibodies and Cancer Therapy. Alan R. Liss. Inc. pp. 77-96).

Techniques described for the production of single chain antibodies, as shown in U.S. Pat. No. 4,946,778, can be adapted to produce single chain antibodies to immunogenic polypeptide products of this invention. Also, transgenic mice may be used to express humanized antibodies to immunogenic polypeptide products of this invention.

It was important to explore the possibility of a test for AFAP1 protein for the diagnosis of Aspergillus infections. In order to examine the subcellular localization of AFAP1 protein, immunogold electron microscopic work was carried out to confirm the potential cell wall localization as suggested by the protein sequence. Example 11 illustrates the cell wall localization of AFAP1 protein.

Detection of Specific Aspergillus Antigen with AFAP1 Specific Antibodies

The specific antibodies can then be used for the detection of Aspergillus antigen from the suspected patients' blood urine or other clinical specimens. Assays used to detect levels of AFAP1 protein in a sample derived from a host are well-known to those of skill in the art and include radioimmunoassays, competitive binding assays, Western Blot analysis, ELISA assays and “sandwich” assay. An ELISA assay (CoLigan. et al., Current Protocols in Immunology, 1(2), Chapter 6, 1991) initially comprises preparing an antibody specific to the AFAP1 antigen, preferably a monoclonal antibody. In addition, a reporter antibody is prepared against the monoclonal antibody. A detectable reagent, such as radioactivity, fluorescence or, in this example, a horseradish peroxidase enzyme, is attached to the reporter antibody. A sample is removed from a host and incubated on a solid support, e.g. a polystyrene dish that binds the proteins in the sample. Any free protein binding sites on the dish are then covered by incubating with a non-specific protein, such as bovine serum albumin. Next, the monoclonal antibody is incubated in the dish during which time the monoclonal antibodies attach to any AFAP1 proteins attached to the polystyrene dish. All unbound monoclonal antibody is washed out with buffer. The reporter antibody linked to horseradish peroxidase is placed in the dish resulting in binding of the reporter antibody to any monoclonal antibody bound to AFAP1. Unattached reporter antibody is then washed out. Peroxidase substrates are then added to the dish and the amount of color developed in a given time period is a measurement of the amount of AFAP1 protein present in a given volume of patient sample when compared against a standard curve.

The ELISA test was set up using purified recombinant AFAP1 protein as indicated in Example 12. The evaluation of such a sandwich test was done first with purified recombinant GST-AFAP1 protein. Using the cell culture media of Aspergillus fumigatus, it was demonstrated that AFAP1 is present in large amount in the culture media of Aspergillus fumigatus cells as shown in Example 1. Finally, as shown in Example 14, it was further demonstrated that circulating AFAP1 antigens can be specifically detected in the serum samples of immunosuppressed patients with invasive aspergillosis. The test is specific since none was shown to be positive for 100 normal blood donors.

A competition assay may be employed wherein antibodies specific to AFAP1 are attached to a solid support. Polypeptides of the present invention are then labeled, for example, by radioactivity, and a sample derived from the host is passed over the solid support. The amount of label detected, for example, by liquid scintillation chromatography, can be correlated to a quantity of AFAP1 in the sample.

A “sandwich” assay is similar to an ELISA assay. In a “sandwich” assay AFAP1 is passed over a solid support and binds to antibody attached to a solid support. A second antibody is then bound to the AFAP1. A third antibody, which is labeled and specific to the second antibody, is then passed over the solid support and binds to the second antibody and the amount can then be quantified.

The levels of AFAP1 antigen in patients' specimens can be used as an indicator of treatment response during the clinical management of invasive aspergillosis. The levels of fungal AFAP1 antigen in patients' blood and urine can be continuously monitored with the above proposed antigen test after the initiation of anti-fungal drug treatment. A decrease in the antigen level can be a good indication of adequate response after treatment. Persistently high levels of the antigen suggests the need for an alternative antifungal drug treatment. An initial decrease followed by a later increase of the AFAP1 antigen level can be indicative of relapse of the infection.

Antibodies to AFAP1 as a Therapeutic Agent

The antibody can also be used for the identification of the pathogenic fungus from tissue biopsies, blood, bone marrow, cerebrospinal fluid, and other specimens of the suspected patients. The presence of AFAP1 specific antigen in the clinical specimens is indicative of current infection in the suspected patients.

The antibody can also be used for therapeutic purpose. It was shown that in infected immunocompetent patients, and in animals that were injected with Aspergillus fumigatus, very high levels of specific antibodies against AFAP1 were detected. These are real reflections of biological responses against the infection by this pathogenic fungus. The administration of high levels of antiserum against AFAP1 may be of significant value as a passive immunization, or therapeutic regimen against the infection by invasive aspergillosis, because it was previously reported that neutropenic patients recovering from invasive aspergillosis had a concomitant increase in anti-Aspergillus antibody titer. Alternatively, humanized monoclonal antibody against AFAP1 can be labeled with a radioisotope, for example, indium-111 or technetium-99 m, and injected into patients with suspected infection. Total body scanning for scintillation could localize the anatomical site of infection as evident by increased uptake. This would facilitate early diagnosis and specific antifungal therapy.

The invention also provides a pharmaceutical pack or kit comprising one or more containers filled with one or more of the ingredients of the pharmaceutical compositions of the invention. Associated with such container(s) can be a notice in the form prescribed by a governmental agency regulating the manufacture, use or sale of pharmaceuticals or biological products, which notice reflects approval by the agency of manufacture, use or sale for human administration. In addition, the pharmaceutical compositions may be employed in conjunction with other therapeutic compounds.

The pharmaceutical compositions may be administered in a convenient manner such as by the intravenous, intraperitoneal, intramuscular, subcutaneous, or intradermal routes. The pharmaceutical compositions are administered in an amount which is effective for treating and/or prophylaxis of the specific indication. In general, the pharmaceutical compositions are administered in an amount of at least about 10 mg/kg body weight, and in most cases they will be administered in an amount not in excess of about 8 mg/kg body weight per day. In most cases, the dosage is from about 10 mg/kg to about 1 mg/kg body weight daily, taking into account the routes of administration, symptoms, etc.

AFAP1 for Immunization

AFAP1 protein can be used for the purpose of immunizing high risk people. Since Aspergillus fumigatus is acquired by inhalation of infectious conidia, immunization could be administered through the mucosal route to stimulate the production of secretary IgA. The specific IgA could have neutralizing activity on the infectious conidia by preventing the adherence of the fungus on to the surface of the host cells that represents the very first step of fungal invasion. Similarly, the immunization with AFAP1 protein can generate cell-mediated immune response that include the activation of helper T cells and cytotoxic T cells. Because AFAP1 produces very high level of antibody response in both animals and infected patients, it is conceivable that the immune response against this specific protein can have protective function against infection.

The immunization can be carried out as recommended by the Immunization Practices Advisory Committee (MMWR 1990;39:1 No. RR-2). Vaccines are given at doses between 10 μg to 1 mg per adult each time. Three injections spaced by one month and six months are suggested. Seroconversion will be tested after the end the final vaccination.

Fragments of the full length AFAP1 gene may be used as a hybridization probe in DNA library screening to isolate similar genes which have high sequence similarities to AFAP1 from many other pathogenic Aspergilus spp. that also cause systemic infection in human. An alternative approach is to generate specific antibodies against AFAP1 and use the antibodies to screen for these genes.

An example of the screening comprises the isolation of a homologous gene by low stringency nucleotide hybridization using the whole or any part of the coding sequence of the AFAP1 gene shown in FIG. 1. Labeled AFAP1 probes having sequences identical or complementary to that of the gene of the present invention are used to screen libraries of other fungal cDNA or genomic DNA libraries to identify and isolate the clones that hybridize to the probe. The hybridization can be carried out at low stringency condition. An example of the condition of hybridization is to incubate the labeled probes with the libraries overnight at 42° C. in a hybridization solution containing 5×SSC, 35% formamide, 5×Denhardts' solution, 250 μg/mL carrier DNA, 50 mM NaPO₄, and 1% SDS. Washes are done twice with 1×SSC. 0.1% SDS at room temperature and twice at 37° C.

Example 6 demonstrates the potential presence of such homologous genes in other Aspergillus species and a method of low stringency hybridization for the detection of such homologous genes in these organisms using the method described above. It is the work of the art to use the above procedure or procedures of similar nature to isolate genes from other pathogenic fungi that are homologous to AFAP1.

Any gene of other infectious fungi identified using the full or any part of the AFAP1 DNA or RNA, protein sequence, or antibodies generated against the AFAP1 protein, will be considered a homologous gene. All the work leading to the identifying and cloning of its homologous genes are considered the work of the art only possible with the knowledge from this invention. Those homologous gene products (recombinant proteins) can then be produced and isolated using the above suggested protocol. The recombinant protein can then be used for serological tests for the presence of specific antibodies and the antigen test in the suspected patients described above. Other applications against all other fungal infections using the AFAP1 gene or homologous genes, including but not limited to all the claims of this invention, are only possible with the knowledge of this invention.

More importantly, this invention also provides a method of using fungus-immunized animal sera for the screening of cDNA expression libraries made from pathogenic fungi. The animals herein specifically included Guinea pig and mouse.

This invention further provides a method of identifying Aspergillus fumigatus Antigenic Protein 1 homologous genes from a fungus other than Aspergillus fumigatus comprising the steps of: a) obtaining a DNA library containing clones having inserts specific for a fungus other than Aspergillus fumigatus; b) hybridizing the clones in the library with at least one specific probe for Aspergillus fumigatus Antigenic Protein 1 gene under conditions permitting hybridization of the specific probe to a Aspergillus fumigatus Antigenic Protein 1 homologous gene; c) isolating clones capable of hybridizing to the probe; d) determining the DNA sequence of the inserts; and e) comparing the homology in sequence between the Aspergillus fumigatus Antigenic Protein 1 gene and the inserts to confirm the identity of the homologous gene. In an embodiment of the above method, it further comprises after step (d) determining the protein sequence encoded by the inserts and comparing the sequence homology of the determined protein sequence with the sequence of Aspergillus fumigatus Antigenic Protein 1.

This invention also provides a method of identifying Aspergillus fumigatus Antigenic Protein 1 homologous genes from a fungus other than Aspergillus fumigatus comprising the steps of: a) obtaining an expression cDNA library containing clones having inserts specific for a fungus other than Aspergillus fumigatus; b) contacting the clones with at least one antibody capable of specifically binding to AFAP1 or its homologous proteins under conditions permitting the binding of said antibody to Aspergillus fumigatus Antigenic Protein 1; c) isolating clones capable of binding to said antibody; d) determining the DNA sequence of the inserts; and e) comparing the homology in sequence between the Aspergillus fumigatus Antigenic Protein 1 gene and the inserts to confirm the identity of the homologous gene which codes for a homologous Aspergillus fumigatus Antigenic Protein 1. In an embodiment of the preceding method, it further comprises after step (d) determining the protein sequence encoded by the inserts and comparing the sequence homology of the determined protein sequence with the sequence of Aspergillus fumigatus Antigenic Protein 1.

This invention provides the isolated Aspergillus fumigatus Antigenic Protein 1 homologous gene identified by the above methods.

This invention also provides nucleic acid molecules of at least 15 nucleotides capable of specifically hybridizing with a unique sequence of the Aspergillus fumigatus Antigenic Protein 1 homologous genes.

This invention provides an isolated Aspergillus fumigatus Antigenic Protein 1 homologous protein. This invention also provides an antibody capable of specifically binding to the Aspergillus fumigatus Antigenic Protein 1 homologous protein.

This invention provides a method for measuring the amount of Aspergillus fumigatus Antigenic Protein 1 homologous protein in a sample comprising the steps of: a) contacting the sample with the antibody capable of specifically binding to the Aspergillus fumigatus Antigenic Protein 1 homologous protein under conditions permitting formation of complexes between said antibody and Aspergillus fumigatus Antigenic Protein 1 homologous protein; and b) measuring the amount of complexes formed in step (a), thereby measuring the homologous protein in the said sample.

This invention also provides a method for determining whether a patient is infected with a particular fungus comprising the steps of: a) obtaining a sample from the patient; and b) contacting the sample with a panel of antibodies composing specific antibodies which are capable of binding to Aspergillus fumigatus Antigenic Protein 1 or Aspergillus fumigatus Antigenic Protein 1 homologous protein such that a positive reaction of a specific antibody will indicate that the patient is infected with a particular fungus.

This invention further provides a method of determining whether a compound is capable of binding to Aspergillus fumigatus Antigenic Protein 1 or a Aspergillus fumigatus Antigenic Protein 1 homologous protein comprising the steps of: a) linking the said protein onto a matrix; b) contacting the compound with the linked protein under conditions permitting formation of complexes between the compound and the polypeptide; and c) detecting the complexes, wherein a positive detection will indicate that the compound is capable of binding to Aspergillus fumigatus Antigenic Protein 1 or a Aspergillus fumigatus Antigenic Protein 1 homologous protein. In an embodiment of this method, the compound is labeled with a detectable marker.

This invention provides a kit for measuring antibodies against Aspergillus fumigatus Antigenic Protein 1 in a sample comprising in separate compartments: a) the polypeptide of an isolated polypeptide selected from the group consisting of a polypeptide comprising amino acid 18 to amino acid 284 of SEQ ID NO: 1, fragments, analogues and derivatives of said polypeptide; and b) a positive control antibody capable of specifically binding to Aspergillus fumigatus Antigenic Protein 1.

This invention also provides a kit for measuring Aspergillus fumigatus Antigenic Protein 1 in a sample composing in separate compartments: a) the antibody capable of specifically binding to the Aspergillus fumigatus Antigenic Protein 1 homologous protein; and b) a positive control of purified Aspergillus fumigatus Antigenic Protein 1.

These diagnostic kits may be based on enzyme-linked immunsorbent assay (ELISA), radioimmunoassay (RIA) or other detection technologies known in the art.

This invention provides a method for identifying a gene coding for an immunogenic protein of a pathogenic fungus comprising the steps of: (a) obtaining the complete DNA sequence of the AFAP1 homologous gene in Aspergillus fumigatus by RACE PCR with primers conserved in homologous PMAP-1 gene of Penicillium marneffei and the homologous IgE binding protein of Aspergillus fumigatus; (b) cloning of the desired PCR fragment into an expression plasmid vector in E. coli; (c) obtaining hyperimmune serum against the whole fungal cells of the pathogen fungus; (d) contacting said hyperimmune serum with the putative clones which contains DNA inserts specific for the pathogen fungus under conditions permitting binding of the expressed insert and said serum; (e) isolating clones capable of binding to said antibody; and (f) determining the DNA sequence of the inserts to identify the genes contained in the clones, wherein the majority of the clones will be coding for this protein of the pathogenic fungus, thereby determining the gene coding for an immunogenic protein of a pathogenic fungus. In an embodiment, the hyperimmune serum is obtained by immunizing Guinea pig with whole fungal cells. Other animals such as rats, mice, or rabbits may be similarly used to produce the hyperimmune serum.

Finally, this invention provides genes identified by the above methods.

The present invention will be further described with reference to the following examples. However, it is to be understood that the present invention is not limited to such examples. All parts or amounts, unless otherwise specified, are by weight.

In order to facilitate understanding of the following examples, certain frequently occurring methods and/or terms will be described.

“Plasmids” are designated by a lower case p preceded and/or followed by capital letters and/or numbers. The starting plasmids herein are either commercially available, publicly available on an unrestricted basis, or can be constructed from available plasmids in accord with published procedures. In addition, equivalent plasmids to those described are known in the art and will be apparent to the ordinarily skilled artisan.

“Digestion” of DNA refers to catalytic cleavage of the DNA with a restriction enzyme that acts only at certain sequences in the DNA. The various restriction enzymes used herein are commercially available and their reaction conditions, cofactors and other requirements were used as would be known to the ordinarily skilled artisan. For analytical purposes, typically 1 mg of plasmid or DNA fragment is used with about 2 units of enzyme in about 20 mL of buffer solution. For the purpose of isolating DNA fragments for plasmid construction, typically 5 to 50 mg of DNA are digested with 20 to 250 units of enzyme in a larger volume. Appropriate buffers and substrate amounts for particular restriction enzymes are specified by the manufacturer. Incubation times of about 1 hour at 37° C. are ordinarily used, but may vary in accordance with the supplier's instructions. After digestion, the reaction is electrophoresed directly on a polyacrylamide gel to isolate the desired fragment.

Size separation of the cleaved fragments is performed using an 8 percent polyacrylamide gel described by Goeddel. D. et al., Nucleic Acids Res, 8:4057 (1980).

“Oligonucleotides” refers to either a single stranded polydeoxynucleotide or two complementary polydeoxynucleotide strands which may be chemically synthesized. Such synthetic oligonucleotides have no 5′ phosphate and thus will not ligate to another oligonucleotide without adding a phosphate with an ATP in the presence of a kinase. A synthetic oligonucleotide will ligate to a fragment that has not been dephosphorylated.

“Ligation” refers to the process of forming phosphodiester bonds between two double stranded nucleic acid fragments (Maniatis T., et al., Id. p. 146). Unless otherwise provided, ligation may be accomplished using known buffers and conditions with 10 units of T4 DNA ligase (“ligase”) per 0.5 mg of approximately equimolar amounts of the DNA fragments to be ligated.

Unless otherwise stated, transformation was performed as described by the method of Graham F and Van der Eb A, Virology 52:456˜57 (1973).

### EXAMPLES

### Example 1

### The cloning of AFAP1 cDNA that Encodes a Highly Antigenic Protein on Aspergillus fumigatus.

The Aspergillus fumigatus strain UPN158, an isolate from a bone marrow transplant recipient, was used throughout the study. One mL suspension of conidia obtained by flushing the surface of Aspergillus fumigatus colonies grown on Sabouraud agar at 37° C. for 4 days was used to inoculate 25 mL Czapek Dox medium (Difco) in 500 mL flask at 37° C. in a gyratory shaker. A 2 day old culture was harvested for RNA extraction.

To construct the cDNA expression library, Stratagene's library construction system was used. Briefly, the Aspergillus fumigatus culture was made as described above. 25 mL of 2 day old mycelia were collected and resuspended in 5 mL TRIzol reagent (GibcoBRL: total RNA isolation reagent). Disruption of mycelia was achieved by sonication. The homogenized samples were incubated for 15-30 minutes at room temperature with rocking. 1 mL of chloroform was added to 5 mL TRIzol reagent. Vortex tubes vigorously for 1 min and incubate the sample further at room temperature for 5 minute The sample was centrifuged at 12,000×g for 15 minutes at 4° C. RNA was removed from the upper aqueous phase and extracted once again with equal volume of chloroform. RNA was precipitated by mixing with 0.5 volume of isopropanol. The sample was incubated at room temperature for 10 minutes and centrifuged at 12,000×g for 10 min at 4° C. The RNA pellet was washed with 75% ethanol and centrifuged at 7,500×g for 5 minutes at 4° C. Dried RNA was dissolved in DEPC-treated water, measured at OD 260, aliquoted and stored at −80° C.

Poly(A) RNA was obtained by using QuickPrep Micro mRNA purification kit (Pharmacia) based on the conventional oligo(dT) cellulose method. Briefly, the RNA sample was mixed with oligo (dT) cellulose resin in guanidinium thiocyanate. The oligo (dT) cellulose was then washed with high salt buffer [10 mM Tris-HCL (pH 7.5), 1 mM EDTA, 0.5 M NaCl, followed by low salt buffer 110 mM Tris-HCl (pH 7.5), 1 mM EDTA, 0.1 M NaCl]. Poly (A)+RNA was eluted with TE buffer [10 mM Tris-HCl (pH 7.5), 1 mM EDTA]. The mRNA was then used for the construction of a lambda-ZAP cDNA expression library (Stratagene. Inc.).

To produce antibody from guinea pig for the screening of the putative cDNA clone, 10 mL of mycelial sediment from a 1-day-old culture was washed 3 times in PBS and finally suspended in 2 mL of PBS containing 0.05% phenol. 500 ul of mycelial suspension was mixed thoroughly with an equal part of complete Freud's adjuvant and injected intramuscularly into the animal's thigh. Incomplete Freud's adjuvant was used in subsequent immunization and a total of 4 inoculations were required to complete the program in 2 months.

Approximately 50,000 plaques from the Aspergillus fumigatus mycelial cDNA library were plated. The phage DNA of the library is extracted and used for screening by RACE-PCR, followed by TA cloning into an expression vector and confirmation by and western blot (Molecular Cloning. Sambrook et al., 1989, Cold Spring Harbor Lab. Press). The procedure was based on the information in the SMART cDNA Amplification kit (ClonTech, Palo Alto, Calif., USA).

Using the DNA and amino acid sequence of PMAP-1 of Penicillium marneffei (see U.S. Pat. No. 5,973,131, dated Oct. 26, 1999), an incomplete DNA sequence called Aspergillus fumigatus mRNA encoding for an unknown protein [GenBank Accession (AA12162 (AJ224865)] was identified in the public database which shares 34% amino acid homology with PMAP-1 at the conserved region of 141 amino acid. Using the nucleotide sequence of this Aspergillus fumigatus protein, PCR primers were constructed for RACE PCR in order to identify the complete gene for cloning. The primers included:

GSP1 (Position 400-376): 5′TGTCGGAGAGGGACTCAGGGACCTT (SEQ ID NO: 4)

NGSP2 (Position 274-250): 5′GGCGACGAACTTGTCCTTCTTGGAG (SEQ ID NO: 5)

GSP2 (Position 80-104): 5′CCGTCTCCTCCTACAACGGTGGTGA (SEQ ID NO: 6)

NGSP3 (Position 265-288): 5′CTCATCTCCAAGAAGGACAAGTTC (SEQ ID NO: 7)

The complete gene was sequenced. It was then amplified by PCR and cloned into PGEX-2T in frame with GST (Clontech, Palo Alto, Cailf., USA). Desired clones were identified as containing the largest gene-specific inserts confirmed by sequencing. Ten fully sequenced clones were picked and induced by 2 mM IPTG. Cellular lysates were extracted and electrophoresed in 10% SDS-polyacrylamide gels. The protein gels were electro-transferred onto nitrocellulose membranes and reacted with sera from preimmunized or immunized guinea pig serum (1:1000), Aspergillus fumigatus infected patients and normal control blood donors (1:500). All ten clones produced a prominent band at about 62 kDa (GST included) position in both immunized guinea pig serum and Aspergillus fumigatus infected-patients serum, but the band was absent in preimmune guinea pig serum and normal blood donor control serum. Further sequence analysis revealed that all ten clones contained the same gene, hereby named Aspergillus fumigatus Antigenic Protein 1 (AFAP1).

### Example 2

### Sequence Analysis of Aspergillus Gene and Identification of the Full Coding Sequence of the Polypeptide.

The sequence was assembled and analyzed by the GCG 8.0 package from Genetics Computer Group, Inc. The result of the sequence analysis is shown in FIG. 1. The AFAP1 gene contains an open reading frame of 284 amino acid residues with a predicted molecular mass of 31.4 kDa. The N-terminal cleavable signal peptide of 17 amino acids is underlined in FIG. 1. The serine/threonine rich region, shown in italics in FIG. 1, indicates that this protein may have many O-glycosylation sites. BLAST analysis was performed using NCBI programs in order to search for potential homologues that might implicate the potential functions of AFAP1. The BLAST search results indicated that AFAP1 has 100% homology to an incomplete gene protein sequence of Aspergillus fumigatus with unknown function. As shown in FIG. 2, examination of AFAP1 protein sequence revealed sequence features that are common for fungal cell wall proteins, including several cell wall proteins of Saccharomyces cerevisiae (Van Der Vaart et al., 1995, J. Bacteriol. 177:3104-3110), and Candida albicans (Bailey D. A. et al. 1996, Bacteriol 178: 5353-5360). AFAP1 has a putative N-terminal signal sequence (underlined/black boxes at N-terminal) found on most secretary proteins (von Heijoe, G. 1986, Nucleic Acid Res., 14:4683-4690), a putative C-terminal glycosylphosphatidylinositol (GPI) domain (underlined/black boxes at C-terminal), and a serine- and threonine-rich region (italic). A GPI domain is utilized by many proteins to anchor them to the eukaryotic cell membrane (Udenfriend, S., 1995, Annu. Rev. Biochem. 64:563-591). Once anchored to the cell membrane, the cell surface proteins can play many important physiological functions, including cell-cell recognition, cell adhesion, receptors, and nutrient and ion transporters. After processing, the mature AFAP1 protein has 249 amino acid residues (without the signal peptide for secretion and the signal peptide for GP1 membrane attachment) with a molecular weight of 24 kDa without glycosylation. AFAP1 is expected to be glycosylated at threonine-serine rich regions between amino acide residue 178 to 259 and 177 to 251 respectively. These stretches of serine/threonine rich region are at its C-terminal half, indicating that the protein should be O-glycosylated.

### Example 3

### In Vitro Translation of the AFAP1 Gene and Immunoprecipitation of the Protein by Immunized Animal Serum.

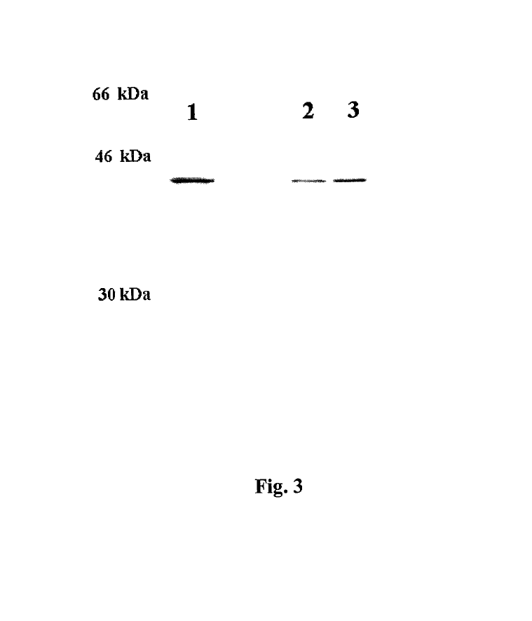

The AFAP1 protein was expressed by the TNT™ Coupled Reticulocyte Lysate System (Promega. Inc.). Briefly, the reaction was set up with 1 μg of pCDNA3 plasmid (subcloned from pAdv-AFAP1), 4 μl of 35S-methionine (1,000 Ci/mmole at 10 mCi/mL), reaction buffer, amino acid mixture minus methionine, RNA polymerase 173 and 25 μl of rabbit reticulocyte lysate. The reaction was incubated at 30° C. for two hours and 0.5 μl of the in vitro translated protein was then analyzed on a 10% SDS-PAGE gel as shown in Lane 1 of FIG. 3.

For immunoprecipitation, 5 μl of the in vitro translated protein was added to 300 μl of lysis buffer containing 50 mM Tris-HCl, pH 7.4, 250 mM NaCl, 0.1% NP40, 5 mM EDTA, 2 μg/μl BSA. 1 μl of each pre-immunized or immunized Guinea pig sera as primary antibodies were added to separated tubes. The incubation was carried out at 4° C. for one hour. 50 μl of 50% Protein G Sepharose (Pharmacia Biotech) was then added to the mixture for a further incubation of one hour at 4° C. on a rocker for constant mixing. The immunocomplex was then pelleted in a microcentrifuge at 4° C. for 5 seconds and washed four times with 1 mL of lysis buffer each. After the final wash the pellet was resuspended into 25 μl of 2×SDS sample buffer and heated to 95° C. for five minutes. 20 μl of the final sample were loaded onto a 10% SDS-PAGE gel. After running, the gel was fixed and then soaked in Amplify™ for 30 minutes (Amersham. Inc.). The gel was dried on a BioRad Gel Dryer (BioRad. Inc.). The film exposure was done at −80° C. with an intensifying screen overnight. Lanes 2 and 3 in FIG. 3 represent immunoprecipitation in-vitro translated AFAP1 by guinea pig serum against whole cell of A. fumigatus and AFAP1 fusion protein, respectively.

The in vitro translation of AFAP1 gene produces a polypeptide of 36 KDa as shown in FIG. 3 consistent with the predicted open reading frame of the AFAP1 gene sequence in the presence of glycosylation. Other minor fragments are the products of the internal translation initiations.

### Example 4

### Specific Recognition of AFAP1 Protein by Sera from Aspergillus fumigatus Infected Patients.

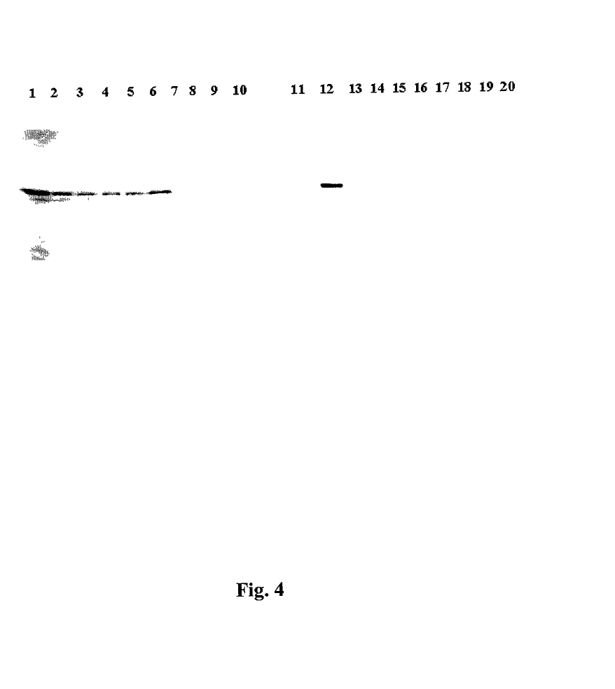

Experiments were done similar to that of Example 3. The results are shown in FIG. 4. Lane 1 represents in-vitro translated AFAP1; lanes 2-20 represent immunoprecipitation of in vitro translated AFAP1 by sera (lanes 2, 12: immune guinea pig sera against AFAP1; lanes 3-6: patients with aspergillosis; lanes 7-10, 17-20: normal blood donors; lane 13-14: patients with candidaemia; lanes 16-17: patients with penicilliosis marneffei). Two Aspergillus fumigatus patients' sera were collected during chemotherapy for haemic malignancies in the Queen Mary Hospital, Hong Kong (Lane 3, 4) They were free from HIV infection and had persistent fever not responsive to antibiotic treatments prior to admission. Two patients were admitted for massive haemoptysis due to an aspergilloma. At postmortem examination and promortem bronchoalveolar lavage, Aspergillus fumigatus was isolated from the abscesses in the lungs with histological evidence of invasion or cavitation. Normal controls were blood samples from normal blood donors that had no history of this fungal infection (Lane 7, 8, 9, 10).

The results show that the patients' sera contained very high levels of anti-AFAP1 antibodies, similar to that of the immunized animal. The immunosuppressed patient serum shows a somewhat lower level of specific antibody but yet significantly higher than that of the normal controls. This result is consistent with the fact that immunosuppressed patients had impaired humoral immune response. The results of this invention indicate that normal control sera do not immunoprecipitate the labeled AFAP1 protein.

### Example 5

### Absence of Immune Cross Reactivity Against AFAP1 Protein from Sera of Other Patients with Fungal Infection.

The experiments were done similar to that of Example 3 and the results are shown in FIG. 4. First, the AFAP1 protein was produced by in vitro translation. The protein was then immunoprecipitated with a panel of antibodies including the ones from patients with systemic fungal infection at the Queen Mary Hospital. Sera from five HIV negative patients with systemic candidiasis, and Penicillium marneffei-infected patients were chosen for the study to determine the specificity of the serological test using AFAP1 protein.

Lane 13 to 17 show no detectable immunoprecipitated band, indicating that these infected patients' sera do not recognize the AFAP1 protein. Therefore, no sera-cross reactivity is observed. Lanes 8, 9, 10 are three other normal control sera of different people from those of Example 4. Again, normal people contain no detectable levels of antibody against this fungal protein.

### Example 6

### Construction of Recombinant Plasmid to Produce GST-AFAP1 Fusion Protein.

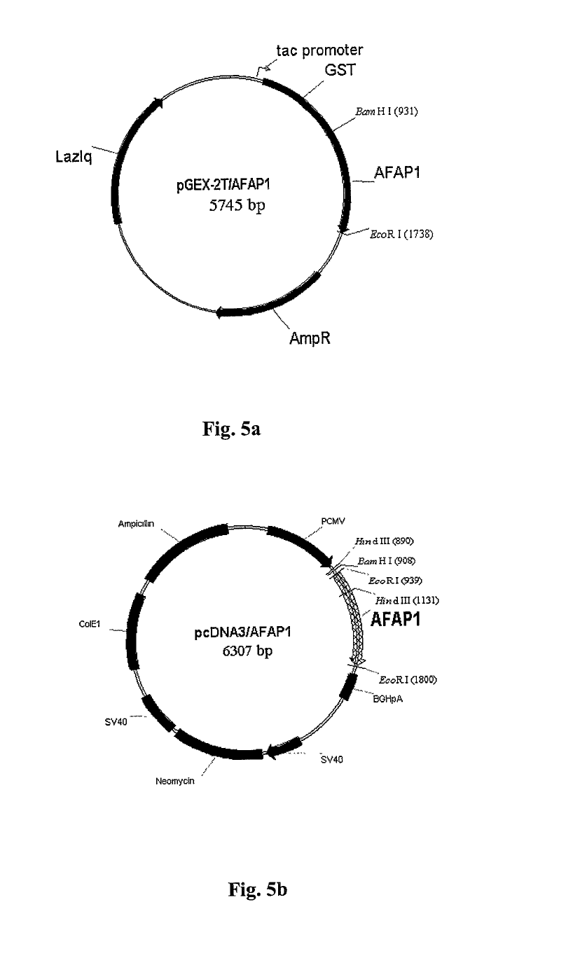

To produce the fusion plasmid for protein purification, primers were used for the amplification of AFAP1 gene from pAdv-AFAP1 plasmid. Amino acid residues 18 to 284 of AFAP1 were amplified and cloned into the BamHI and ECORI sites of a prokanyotic pGEX-2T expression vector in frame, and downstream, of glutathione S-transferase. The map of such a plasmid is shown in FIG. 5a. Similarly, the gene was cloned in pcDNA3 downstream of the CMV promoter. The map of such a plasmid is shown in FIG. 5b.

### Example 7

### Expression and Purification of Recombinant AFAP1 Protein in E. coli.

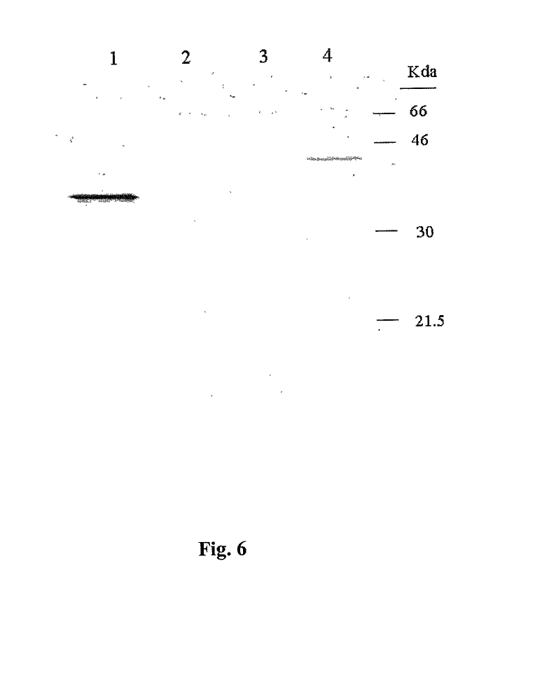

E. coli cells carrying the GST-AFAP1 plasmid were induced with 1 mM of IPTG to express the fusion protein. The GST-AFAP1 fusion protein was expressed and purified as described by GST Gene Fusion System (Pharmacia Biotech). 10 to 15 mg of protein were routinely obtained from 1 litre of E. coli cells carrying the fusion plasmid. The purified fusion protein was cleaved with thrombin and separated on an SDS gel followed by Coomassie blue staining. After purification, a band of 32 kDa can be seen on the SDS gel, consistent with the expected molecular weight for the fusion protein of 33 kDa. To confirm the purified fusion protein as GST-AFAP1, Western blot analysis of the purified and cleaved fusion protein was carried out using serum from Guinea pig that was immunized by killed Aspergillus fumigatus cells. The results indicated that the purified proteins are highly reactive to the Guinea pig immune serum against the killed Aspergillus fumigatus (lane 1, FIG. 6). When a eukaryotic expression system is used, a band of 36 kDa is visible after SDS separation and western blot analysis. FIG. 6 shows Western blot analysis of the recombinant AFAP1 protein. Lane 1: The sequence coding amino acid residues 19 to 284 of AFAP1 protein was expressed in prokaryotic vector pGEX2T. Lane 2: supernatant from 293 cells which were transfected with eukaryotic vector pSecTag2 encoding MP1 (recombinant protein of Penicillium marneffei). Lane 3: supernatant from 293 cells which were transfected with eukaryotic vector pcDNA3 encoding full-length sequence of AFAP1. Lane 4: 293 cells lysate were transfected with eukaryotic vector pcDNA3 encoding full-length sequence of AFAP1.

### Example 8

### Production of Anti-AFAP1 Specific Antibodies.

To produce the antibody from Guinea pig for the screening of the expression library, mycelial cells from 10 mL of a 2-day-old culture were washed 3 times in 2 mL PBS. The mycelial suspension was mixed thoroughly with an equal part of complete Freud's adjuvant and injected intramuscularly into the animal's thigh. Incomplete Freud's adjuvant was used in subsequent immunization and a total of 4 inoculations were required to complete the program in 2 months.

To produce specific antibodies against AFAP1p, 500 μg/250 μg of GST-AFAP1p recombinant protein were mixed with equal parts of complete Freud's adjuvant and injected subcutaneously into two rabbits and three Guinea pigs. Incomplete Freud's adjuvant was used in subsequent injections. Serum was taken two weeks after the third injections.

### Example 9

### AFAP1 is Specifically Located in the Cell Wall of Aspergillus fumigatus.

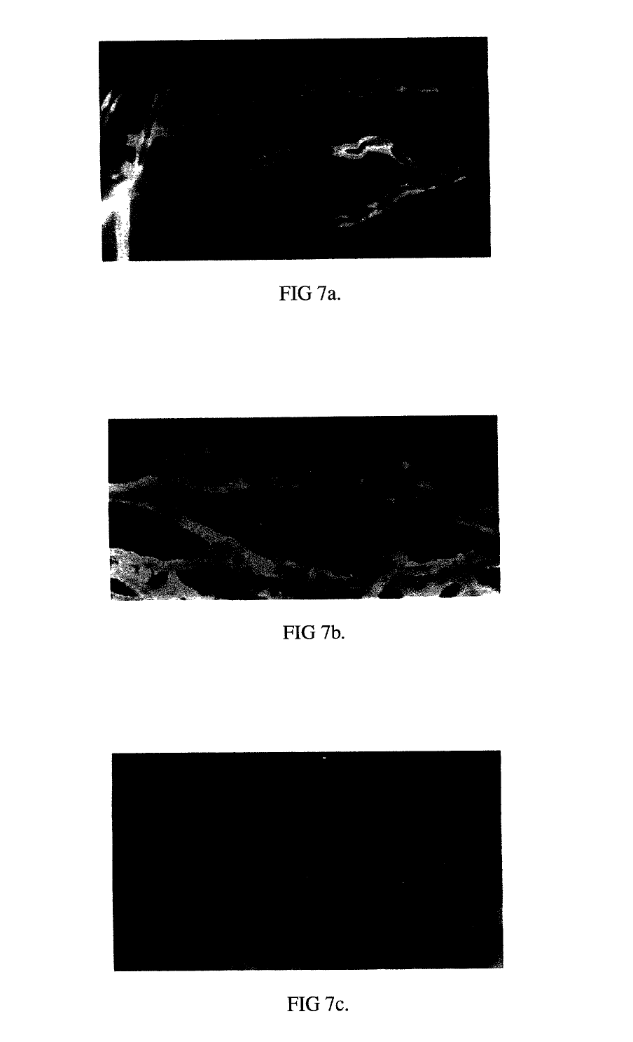

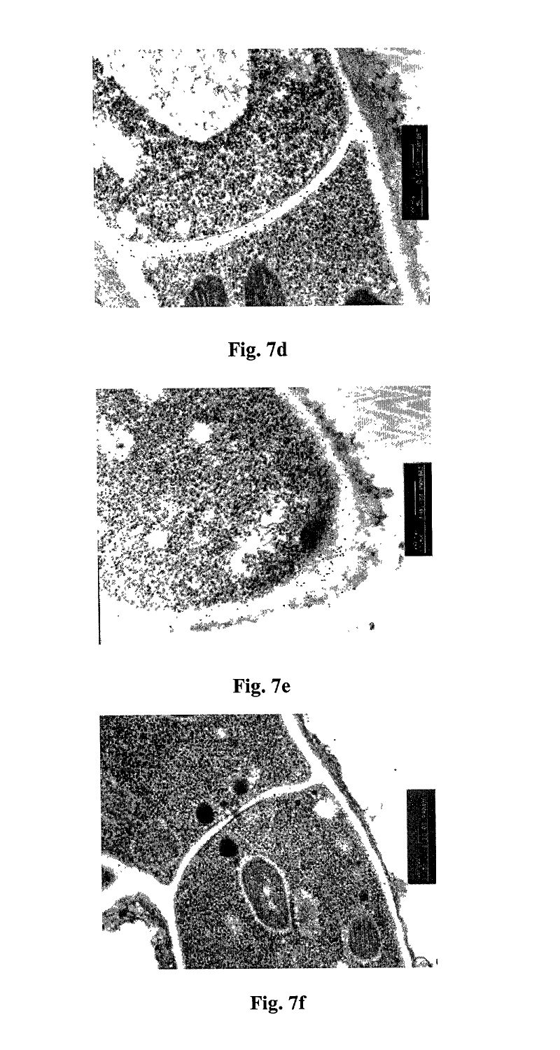

In the indirect immunofluorescent assay, Aspergillus fumigatus mold cells were harvested and washed twice in PBS. Cells were deposited on teflon-coated slides, air-dried, and fixed in cold acetone for 10 min. Rabbit serum with antibodies specific against AFAP1 were added to the fixed cells and incubated in a humidity chamber at 37° C. for 45 min. A rabbit serum with antibodies against whole cells of Aspergillus fumigatus and a preimmuine rabbit serum were used as the positive and negative controls, respectively. The cells were then washed with PBS, air-dried, and incubated with affinity-purified fluorescein isothiocyanate-conjugated anti-rabbit IgG (DAKO A/S, Denmark) at 37° C. for 45 min. The cells were mounted and observed under ultraviolet light. Indirect immunofluorescent staining for AFAP1 by monospecific guinea pig sera against AFAP1 showed that the protein is located in the outer covering of the mycelia (FIG. 7a). A positive control using guinea pig anti-sera against whole cells of Aspergillus fumigatus showed diffuse fluorescence (FIG. 7b). A negative control using non-immune guinea pig sera showed no fluorescence (FIG. 7c). The presence of green fluorescence denotes the presence of antigens being detected by the specific antibodies whereas a red fluorescence denotes a negative reaction. The pattern of fluorescence shown in FIG. 7a is indicative of the presence of AFAP1 on the cell wall of the mycelia.

Immunogold staining of Aspergillus fumigatus cells with anti-AFAP1 antibody was carried out and the electron microscopic results are shown in FIG. 7 (d, e). Staining of Aspergillus fumigatus mycelia with specific rabbit anti-AFAP1 antibody showed that the protein is specifically located at the inner layer of the mycelial cell wall throughout the entire cell, including the tip and septum. Negative control staining of Aspergillus fumigatus mycelia revealed no specific staining of the cell (FIG. 7f).

### Example 10

### Antibody Detection Test for Anti-AFAP1 Antibodies.

AFAP1 Antibody Test

| | | |
|---|---|---|
| Coating buffer | For 1 liter: | pH 9.6 |
| | Na₂CO₃ | 1.5 g |
| | NaHCO₃ | 2.93 g |
| Blocking buffer | 2% BSA/PBS | pH 7.4 |
| Wash buffer | PBS/0.05% Tween 20 | pH 5.0 |
| Substrate buffer | For 1 L | pH 5.0 |
| | Citric acid·H₂O | 7.3 g |
| | Na₂HPO₄·12H₂O | 23.88 g |

Plates were coated the night before with 1:2000 dilution of AFAP1 protein (10 mg/mL) in coating buffer overnight at 4° C. Wash plate 2 times with wash buffer. Block plate with blocking buffer using 100 mL/well. Incubate at 37° C. for 1 hour. Dilute serum sample 1:200 and 1:400 in blocking buffer. Add 100 μl of diluted sample into each well. Incubate in 37° C. for 1 hour. Wash plate 4 times with wash buffer. Dilute conjugate 1:7000 in blocking buffer. Add 100 μl of diluted conjugate into each well. Incubate at 37° C. for 1 hour.

Dissolve 4 OPD tablets into 12 mL of substrate buffer. Before use, add 5 μl of 30% H₂O₂ into above mix. Wash plate 4 times as above. Add 100 μl of above substrate mix into each well. Develop at 37° C. until signal appears or for 30 minutes.

Stop reaction by adding 25 μl of 2M H₂SO₄ into each well. Read OD as soon as possible.

Measurement filter 492 nm.

Reference filter 405 nm.

### Example 11

### Detection of the Presence of Specific Antibodies Against AFAP1 Protein in Aspergillus fumigatus Infected Patients.

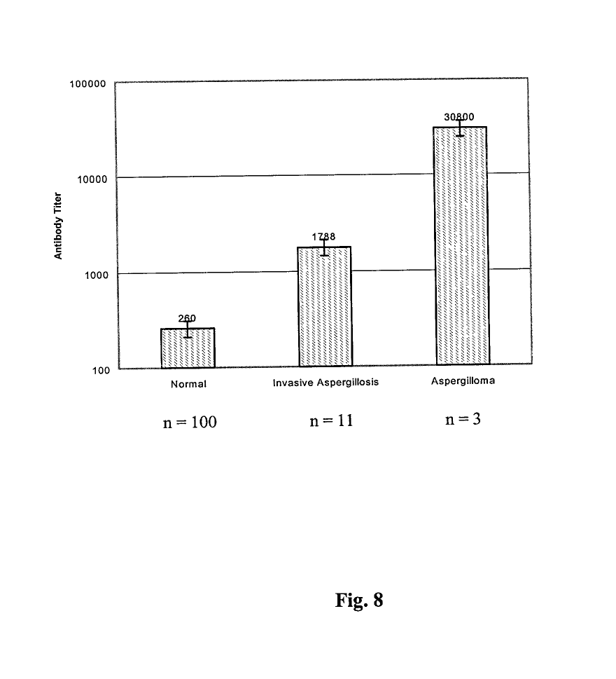

An evaluation of the ELISA test for the presence of anti-AFAP1 antibody is shown in FIG. 8. The numbers used for the evaluation are: 1) 100 blood donors as the normal control; 2) 11 immunocompromised patients with invasive aspergillosis, including patients with haemic malignancies, chronic granulomatous disease of childhood, and solid organ transplants; and 3) 3 specimens from patients with aspergilloma. Antibody titers were the reverse of the highest dilution where the OD readings were still no less than 1.0.

The results shown in FIG. 8 indicate that high level of specific antibodies against AFAP1 were detected in immunocompetent patients with aspergilloma.

### Example 12

### Antigen Detection Test for the Detection of AFAP1 Protein.

AFAP1 Antigen Test

| | | |
|---|---|---|
| Coating buffer | For 1 liter: | pH 9.6 |
| | Na₂CO₃ | 1.5 g |
| | NaHCO₃ | 2.93 g |
| Blocking buffer | 2% BSA/PBS | pH 7.4 |
| Wash buffer | PBS/0.05% Tween 20 | pH 7.4 |
| Substrate buffer | For 1 L | pH 9.8 |
| | Glycine | 7.51 g |
| | MgCl₂ | 203 mg |
| | ZnCl₂ | 136 mg |

Coat plate with Guinea pig anti-AFAP1 antibody (1:5000) in coating buffer 100 μl/well overnight at 4° C. Wash plates 2 times with washing buffer. Block plates with 100 μl/well of blocking buffer. Incubate for 1 hr at 37° C. Dilute AFAP1 protein standard and samples in blocking buffer. Incubate for 1 hr at 37° C. Wash plates 4 times with washing buffer. Dilute rabbit anti-AFAP1 (1:500) in blocking buffer and add 100 μl/well. Incubate for 1 hr at 37° C. Wash plates 4 times as above. Dilute goat anti-rabbit alkaline phosphatase conjugate 1:2000 in blocking buffer and add 100 μl/well. Incubate for 1 hr at 37° C. Wash plates 4 times as above. Dissolve 2 p-nitrophenyl phosphate (pNPP) tablets in 10 mL of substrate buffer. Add 100 μl/well and incubate at 37° C. until color develops. Stop by adding 25 μl/well 3N NaOH into each well. Read OD at 405 nm as soon as possible.

### Example 13

### Detection of the Presence of Specific AFAP1 Protein Antigen in Aspergillus fumigatus Cell Culture Medium.

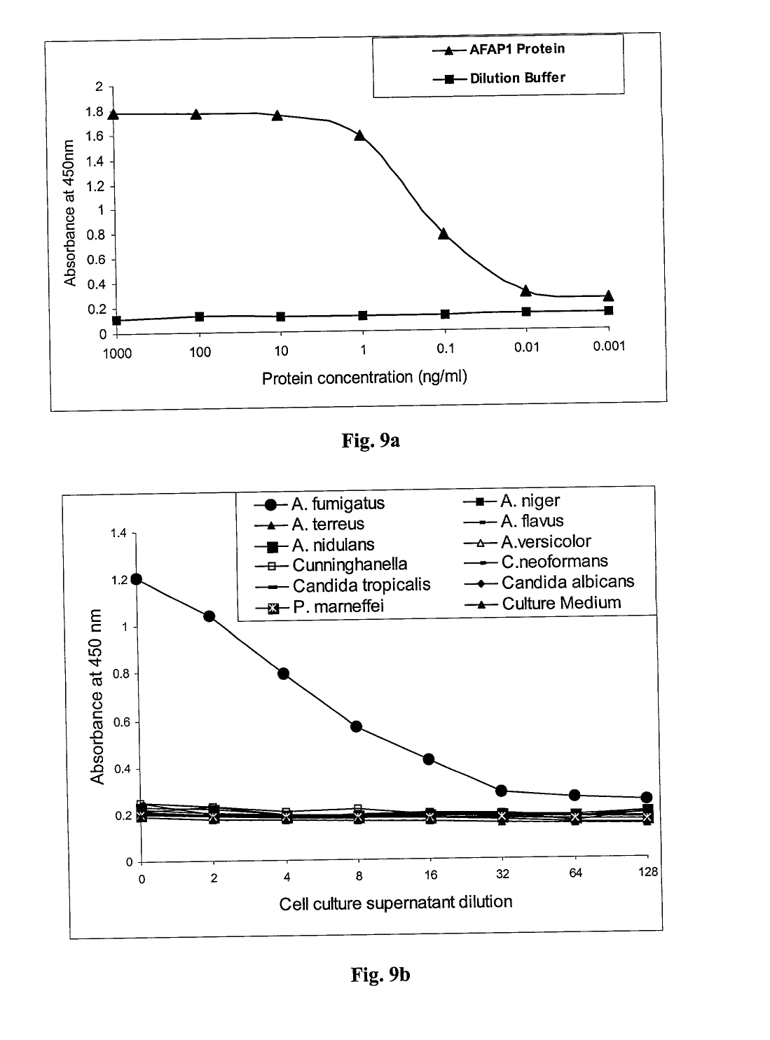

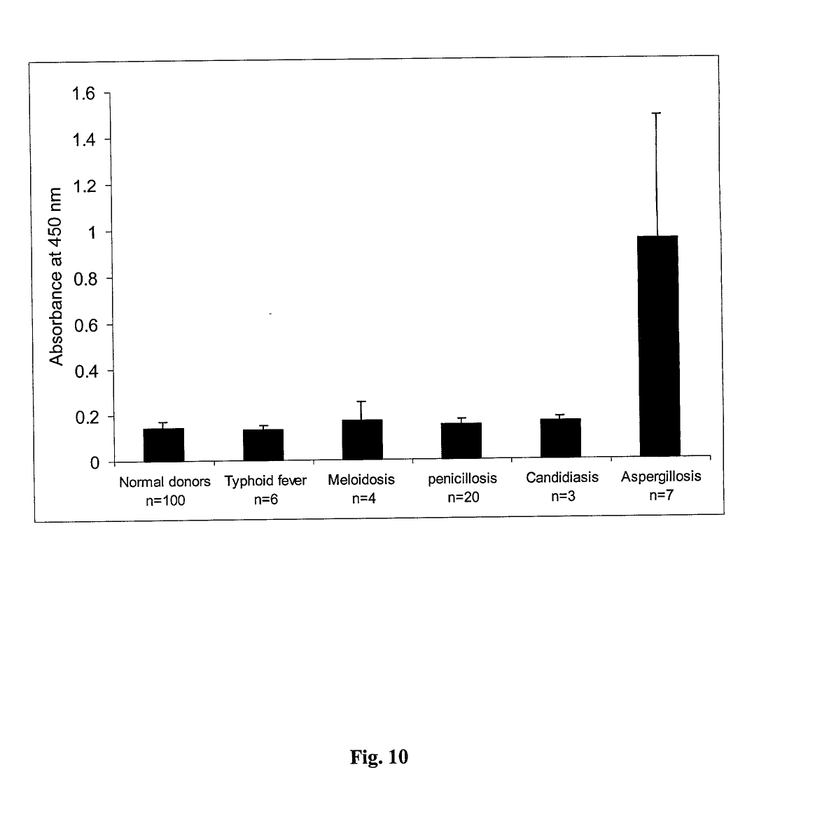

Aspergillus fumigatus cell cultures were obtained by inoculating 10⁶ conidia into 10 mL of Czapek Dox broth incubated at 37° C. for 48 hr. The media was harvested and filtered through a 0.45 μm filter to remove all mycelia. An ELISA test was performed with the media based on the description of Example 12. The standard curve was established in the same experiment with the purified recombinant AFAP1 protein that had been quantitated based on the Bradford Assay (Bio-Rad. Inc.). The result of the study is shown in FIG. 9a.

The sensitivity of the AFAP1 antigen test is 1 pg/test for the stock solution where 100 μl was used for each well on the ELISA plates. The culture media from overnight mycelia contain approximately 1 μg/mL of AFAP1 protein, or about 1000 greater than the minimal sensitivity of the test. The result indicated that AFAP1 protein was shed into the culture media in large quantities. The specificity of the study is illustrated in FIG. 9b. No AFAP1 antigen is found in the supernatant of other fungal cultures.

### Example 14

### Detection of the Presence of Specific-AFAP1 Protein Antigen in Aspergillus fumigatus Infected Patients.

A clinical evaluation of the ELISA test for the presence of AFAP1 antigen is shown in FIG. 10. The numbers used for the evaluation are: 1) 100 blood donors as the normal control; 2) two specimens from immunocompetent patients with aspergilloma; and 3) nine specimens from immunosuppressed patients with invasive aspergillosis. To determine the exact levels of AFAP1 protein in patients sera, a standard curve was established in the same experiment similar to that described in Example 13.

The test allows the detection of circulating AFAP1 protein antigen in immunosuppressed patients at a concentration of 10 ng/mL, approximately 6 times higher than the limit of the sensitivity. The test is specific with not a single positive sera from 100 normal blood donors.

Although preferred embodiments of the invention have been shown and described, it should be understood that various modifications and substitutions, as well as rearrangements and combinations, can be made by those skilled in the art, without departing from the spirit and scope of this invention.

What is claimed is:

1. An isolated polynucleotide comprising a member selected from the group consisting of:
(a) a polynucleotide of at most 852 nucleotides encoding the polypeptide as set forth in SEQ ID NO: 1;
(b) a polynucleotide capable of selectively hybridizing to the polynucleotide of (a); and
(c) a polynucleotide comprising a nucleotide sequence fully complementary to the polynucleotide of (a) or (b).

2. The isolated polynucleotide of claim 1, wherein said member is (a).

3. The isolated polynucleotide of claim 2, wherein said member is (a) and the polypeptide comprises amino acid 1 to 284 of SEQ ID NO. 1.

4. The isolated polynucleotide of claim 1, wherein the polynucleotide is DNA.

5. A method of making a recombinant vector comprising inserting the isolated polynucleotide of claim 2 into a vector wherein said polynucleotide is DNA.

6. A recombinant host cell comprising the polynucleotide of claim 2, wherein said polynucleotide is DNA.

7. A method for producing a polypeptide comprising expressing from the recombinant cells of claim 6, the polypeptide encoded by said polynucleotide and isolated said polypeptide.

8. A process for producing a polypeptide comprising expressing from a recombinant cell containing the polynucleotide of claim 4 the polypeptide encoded by said polynucleotide and isolating said polypeptide.

9. A method of identifying Aspergillus fumigatus Antigenic Protein 1 (SEQ ID NO.: 1) homologous genes from Aspergillus species comprising steps of:
(a) obtaining a DNA library containing clones having inserts specific for a fungus other than Aspergillus fumigatus;
(b) hybridizing the clones in the library with at least one specific probe for Aspergillus fumigatus Antigenic Protein 1 gene under conditions permitting hybridization of the specific probe to a Aspergillus fumigatus Antigenic Protein 1 homologous gene;
(c) isolating clones that hybridize to the probe;
(d) determining the DNA sequence of the inserts;
(e) comparing the homology in sequence between the Aspergillus fumigatus Antigenic protein 1 gene and the inserts to confirm the identity of the homologous gene.

10. An isolated polypeptide selected from the group consisting of a polypeptide comprising amino acid 1 to amino acid 284 of SEQ ID NO: 1, fragments and derivatives of said polypeptide.

11. An antibody, either polyclonal or monoclonal, capable of specifically binding to the polypeptide of claim 9.

12. A method for measuring the level of specific antibodies that reacts with AFAP1 in a sample comprising steps of:
a) containing the sample with the polypeptide of claim 10 under conditions permitting formation of a complex between the AFAP 1 and the antibodies; and
b) measuring the amount of the complexes formed in step (a), thereby measuring the specific antibodies in said sample.

13. The method of claim 12, where used with clinical specimens, such as serum, or blood samples, can generate results that are useful for the diagnosis of infectious diseases caused by Aspergillus species. The diseases may include both aspergilloma and aspergillosis.

14. A method for measuring the presence and the level of AFAP1 protein or protein fragments that react with the specific antibodies specified in claim 11 in a sample comprising steps of:
a) containing the sample with the antibody of claim 10 under conditions permitting formation of a complex between the AFAP 1 and the antibody; and
b) measuring the amount of the complexes formed in step (a), thereby measuring the amount of AFAP1 or protein fragments in said sample.

15. The method of claim 14, where used with clinical specimens, such as serum, blood, tissue samples, or culture from these specimens, can generate results that are useful for the diagnosis of infectious diseases caused by Aspergillus species. The diseases may include both aspergilloma and aspergillosis.

16. The method of claim 14 can generate results that are indicator for monitoring the treatment response for anti-fungal therapy for patients with aspergilloma and aspergillosis.

17. A method of immunization with the isolated AFAP1, its fragments or derivatives of claim 10.

18. The method of claim 17 can be used to reduce or prevent the occurrence of invasive aspergillosis in the immunocompromised patients, including patients with bone marrow transplant or solid organ transplant.
